(19) United States
(12) Patent Application Publication
(10) Pub. No.: US 2008/0021012 A1
(43) Pub. Date: Jan. 24, 2008

(54) 3-[4-{6-Substituted Alkanoyl Pyridin-3-Yl}-3-Phenyl]-5-(1H-1,2,3-Triazol-1-Ylmethyl)-1,3-Oxazolidin-2-Ones As Antibacterial Agents

(75) Inventors: Michael Barry Gravestock, Holliston, MA (US); Folkert Reck, Sudbury, MA (US); Fei Zhou, Southborough, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 11/569,408

(22) PCT Filed: May 24, 2005
(86) PCT No.: PCT/GB05/02051

§ 371 Date: Nov. 20, 2006

(30) Foreign Application Priority Data
May 25, 2004 (GB) 0411593.7
Jan. 5, 2005 (GB) 0500054.2

(51) Int. Cl. A61K 31/397; A61K 31/4439; A61K 31/4523; A61K 31/5377; A61P 31/04; C07D 205/02; C07D 213/02; C07D 221/00; C07D 295/00

(52) U.S. Cl. 514/210.18; 514/236.2; 514/253.1; 514/318; 514/340; 514/341; 544/124; 544/364; 546/193; 546/271.4

(57) ABSTRACT

Compounds of formula (I) as well as pharmaceutically-acceptable salts and pro-drugs thereof are disclosed wherein R¹, R², R³, and R⁴ are defined herein. Also disclosed are processes for making compounds of formula (I) as well as methods of using compounds of formula (I) for treating bacterial infections.

The present invention relates to antibiotic compounds and in particular to antibiotic compounds containing substituted oxazolidinone rings. This invention further relates to processes for their preparation, to intermediates useful in their preparation, to their use as therapeutic agents and to pharmaceutical compositions containing them.

The international microbiological community continues to express serious concern that the evolution of antibiotic resistance could result in strains against which currently available antibacterial agents will be ineffective. In general, bacterial pathogens may be classified as either Gram-positive or Gram-negative pathogens. Antibiotic compounds with effective activity against both Gram-positive and Gram-negative pathogens are generally regarded as having a broad spectrum of activity. The compounds of the present invention are regarded as effective against both Gram-positive and certain Gram-negative pathogens.

Gram-positive pathogens, for example Staphylococci, Enterococci, Streptococci and mycobacteria, are particularly important because of the development of resistant strains which are both difficult to treat and difficult to eradicate from the hospital environment once established. Examples of such strains are methicillin resistant staphylococcus (MRSA), methicillin resistant coagulase negative staphylococci (MRCNS), penicillin resistant Streptococcus pneumoniae and multiply resistant Enterococcus faecium.

The major clinically effective antibiotic for treatment of such resistant Gram-positive pathogens is vancomycin. Vancomycin is a glycopeptide and is associated with various toxicities including nephrotoxicity. Furthermore, and most importantly, antibacterial resistance to vancomycin and other glycopeptides is also appearing. This resistance is increasing at a steady rate rendering these agents less and less effective in the treatment of Gram-positive pathogens. There is also now increasing resistance appearing towards agents such as β-lactams, quinolones and macrolides used for the treatment of upper respiratory tract infections, also caused by certain Gram negative strains including H. influenzae and M. catarrhalis.

Certain antibacterial compounds containing an oxazolidinone ring have been described in the art (for example, Walter A. Gregory et al in J. Med. Chem. 1990, 33, 2569-2578 and 1989, 32(8), 1673-81; Chung-Ho Park et al in J. Med. Chem. 1992, 35, 1156-1165). Bacterial resistance to known antibacterial agents may develop, for example, by (i) the evolution of active binding sites in the bacteria rendering a previously active pharmacophore less effective or redundant, and/or (ii) the evolution of means to chemically deactivate a given pharmacophore, and/or (iii) the evolution of efflux pathways. Therefore, there remains an ongoing need to find new antibacterial agents with a favourable pharmacological profile, in particular for compounds having useful activity and physicochemical properties.

Physicochemical properties (such as solubility and bioavailability) of a pharmaceutical compound are generally understood to be a balance between the polarity of the various substituents on the compound, and factors such as molecular weight (with higher molecular weight generally decreasing solubility and bioavailability for equivalent polarity). Other factors such as the rigidity/flexibility of a molecule also generally affect physicochemical properties such as solubility.

Patent application WO 01/94342 (Dong A. Pharm. Co. Ltd) describes pyridyl- or pyrimidyl-phenyl-oxazolidinone compounds bearing a methylacetamide side chain attached to the oxazolidinone ring. The majority of the compounds exemplified in that patent application contain substituted piperazine rings attached to the pyridyl or pyrimidyl ring, or contain other heterocycles such as piperidine, oxadiazole or tetrazole rather than piperazine.

We have discovered a novel group of pyridyl-phenyl-oxazolidinone compounds, bearing a triazole substituent on the oxazolidinone ring, and acyclic substituents directly linked to the pyridyl ring, which have useful antibacterial activity.

The compounds of this invention generally have good physical and/or pharmacokinetic properties, for example solubility and bioavailability.

Furthermore, the compounds of the invention generally have favourably low mono-amine oxidase-A inhibition.

Accordingly the present invention provides a compound of the formula (I), or a pharmaceutically-acceptable salt, or pro-drug thereof,
wherein: 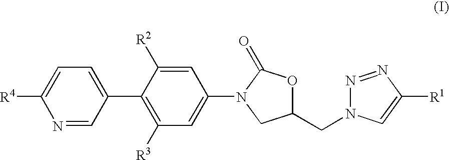
R¹ is selected from hydrogen, halogen, cyano, methyl, cyanomethyl, fluoromethyl, difluoromethyl, trifluoromethyl, methylthio, and (2-4C)alkynyl;
R² and R³ are independently selected from hydrogen, fluoro, chloro and trifluoromethyl;
R⁴ is —C(O)R⁵; or
R⁴ is selected from —C(H)═N—OR⁸, —C(R⁵)═N—OH and —C(R⁵)═N—OR⁸;
R⁵ is (1-6C)alkyl (substituted with 1 or 2 substituents independently selected from hydroxy, carboxy, (1-4C)alkoxy, HET-1 and NR⁶R⁷);
or R⁵ is (3-6C)cycloalkyl (optionally substituted with 1 substituent selected from hydroxy, carboxy, (1-4C)alkoxy and NR⁶R⁷);
or R⁵ is HET-1;
R⁶ and R⁷ are independently selected from hydrogen, methyl, cyclopropyl (optionally substituted with methyl), carboxymethyl and (2-4C)alkyl (optionally substituted by a substituent selected from amino, (1-4C)alkylamino, di-(1-4C)alkylamino, carboxy, (1-4C)alkoxy and hydroxy);
or R⁶ and R⁷ together with a nitrogen to which they are attached form a 4, 5 or 6 membered, saturated or partially unsaturated heterocyclyl ring, optionally containing 1 further heteroatom (in addition to the linking N atom) independently selected from O, N and S, wherein a —CH₂-group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen to which R⁶ and R⁷are attached is not thereby quaternised) by 1 or 2 (1-4C)alkyl groups;
or R⁶ and R⁷ together with a nitrogen to which they are attached form an imidazole ring, which ring is optionally substituted on an available carbon atom by 1 or 2 (1-4C)alkyl (wherein a (1-4C)alkyl group is optionally substituted by methoxy or ethoxy);
R⁸ is (1-6C)alkyl (optionally substituted with 1 or 2 substituents independently selected from hydroxy, carboxy, (1-4C)alkoxy and NR⁶R⁷);
HET-1 is a 4, 5 or 6 membered saturated or partially unsaturated heterocyclyl ring, containing 1 or 2 heteroatoms independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen is not thereby quaternised) by 1 or 2 (1-4C)alkyl.

In a further aspect of the invention, there is provided a compound of formula (I) as hereinbefore defined wherein
R⁶ and R⁷ together with a nitrogen to which they are attached form an imidazole ring, which ring is optionally substituted on an available carbon atom by 1 or 2 (1-4C)alkyl; and
HET-1 is a 5- or 6-membered saturated or partially unsaturated heterocyclyl ring.

In another aspect, the invention relates to compounds of formula (I) as hereinabove defined or to a pharmaceutically acceptable salt.

In another aspect, the invention relates to compounds of formula (I) as hereinabove defined or to a pro-drug thereof. Suitable examples of pro-drugs of compounds of formula (I) are in-vivo hydrolysable esters of compounds of formula (I). Therefore in another aspect, the invention relates to compounds of formula (I) as hereinabove defined or to an in-vivo hydrolysable ester thereof.

It will be understood that a 4, 5 or 6 membered, saturated or partially unsaturated heterocyclyl ring containing 1 or 2 heteroatoms independently selected from O, N and S (whether or not one of those heteroatoms is a linking N atom), as defined in any definition herein (for example the definiton of HET-1), does not contain any O—O, O—S or S—S bonds.

Where optional substituents are chosen from “0, 1, 2 or 3” groups it is to be understood that this definition includes all substituents being chosen from one of the specified groups or the substituents being chosen from two or more of the specified groups. An analogous convention applies to substituents chose from “0, 1 or 2” groups and “1 or 2” groups.

In this specification the term ‘alkyl’ includes straight chained and branched structures. For example, (1-4C)alkyl includes propyl and isopropyl. However, references to individual alkyl groups such as “propyl” are specific for the straight chained version only, and references to individual branched chain alkyl groups such as “isopropyl” are specific for the branched chain version only. A similar convention applies to other radicals, for example halo(1-4C)alkyl includes 1-bromoethyl and 2-bromoethyl. In this specification, the terms ‘alkenyl’ and ‘cycloalkenyl’ include all positional and geometrical isomers.

Within this specification composite terms are used to describe groups comprising more than one functionality such as (1-4C)alkoxy-(1-4C)alkoxy-(1-4C)alkyl. Such terms are to be interpreted in accordance with the meaning which is understood by a person skilled in the art for each component part. For example (1-4C)alkoxy-(1-4C)alkoxy-(1-4C)alkyl includes methoxymethoxymethyl, ethoxymethoxypropyl and propoxyethoxymethyl.

It will be understood that where a group is defined such that is optionally substituted by more than one substituent, then substitution is such that chemically stable compounds are formed. For example, a trifluoromethyl group may be allowed but not a trihydroxymethyl group. This convention is applied wherever optional substituents are defined.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

Examples of (1-4C)alkyl include methyl, ethyl, propyl, isopropyl and t-butyl; examples of (2-4C)alkyl include ethyl, propyl, isopropyl and t-butyl; examples of (1-6C)alkyl include methyl, ethyl, propyl, isopropyl, t-butyl, pentyl and hexyl; examples of (3-6C)alkyl include propyl, isopropyl, t-butyl, pentyl and hexyl; examples of hydroxy(1-4C)alkyl include hydroxymethyl, 1-hydroxyethyl, 2-hydroxyethyl and 3-hydroxypropyl; examples of hydroxy(2-4C)alkyl include 1-hydroxyethyl, 2-hydroxyethyl, 2-hydroxypropyl, 3-hydroxypropyl, 1-hydroxyisopropyl and 2-hydroxyisopropyl; examples of (1-4C)alkoxycarbonyl include methoxycarbonyl, ethoxycarbonyl and propoxycarbonyl; examples of (2-4C)alkenyl include allyl and vinyl; examples of (2-4C)alkynyl include ethynyl and 2-propynyl; examples of (1-4C)alkanoyl include formyl, acetyl and propionyl; examples of (1-4C)alkoxy include methoxy, ethoxy and propoxy; examples of (1-6C)alkoxy and (1-10C)alkoxy include methoxy, ethoxy, propoxy and pentoxy; examples of (1-4C)alkylthio include methylthio and ethylthio; examples of (1-4C)alkylamino include methylamino, ethylamino and propylamino; examples of di-((1-4C)alkyl)amino include dimethylamino, N-ethyl-N-methylamino, diethylamino, N-methyl-N-propylamino and dipropylamino; examples of halo groups include fluoro, chloro and bromo; examples of (1-4C)alkoxy-(1-4C)alkoxy and (1-6C)alkoxy-(1-6C)alkoxy include methoxymethoxy, 2-methoxyethoxy, 2-ethoxyethoxy and 3-methoxypropoxy; examples of (1-4C)alkanoylamino and (1-6C)alkanoylamino include formamido, acetamido and propionylamino; examples of (1-4C)alkylS(O)q- wherein q is 0, 1 or 2 include methylthio, ethylthio, methylsulfinyl, ethylsulfinyl, methylsulfonyl and ethylsulfonyl; examples of hydroxy-(2-4C)alkoxy include 2-hydroxyethoxy and 3-hydroxypropoxy; examples of (1-6C)alkoxy-1-6C)alkyl and (1-4C)alkoxy(1-4C)alkyl include methoxymethyl, ethoxymethyl and propoxyethyl; examples of (1-4C)alkylcarbamoyl include methylcarbamoyl and ethylcarbamoyl; examples of di((1-4C)alkyl)carbamoyl include di(methyl)carbamoyl and di(ethyl)carbamoyl; examples of halo groups include fluoro, chloro and bromo; examples of halo(1-4C)alkyl include, halomethyl, 1-haloethyl, 2-haloethyl, and 3-halopropyl; examples of dihalo(1-4C)alkyl include difluoromethyl and dichloromethyl; examples of trihalo(1-4C)alkyl include trifluoromethyl; examples of amino(1-4C)alkyl include aminomethyl, 1-aminoethyl, 2-aminoethyl and 3-aminopropyl; examples of cyano(1-4C)alkyl include cyanomethyl, 1-cyanoethyl, 2-cyanoethyl and 3-cyanopropyl; examples of (1-4C)alkanoyloxy include acetoxy, propanoyloxy; examples of (1-6C)alkanoyloxy include acetoxy, propanoyloxy and tert-butanoyloxy; examples of (1-4C)alkylaminocarbonyl include methylaminocarbonyl and ethylaminocarbonyl; examples of di((1-4C)alkyl)aminocarbonyl include dimethylaminocarbonyl and diethylaminocarbonyl.

Where optional substituents are listed such substitution is preferably not geminal disubstitution unless stated otherwise. If not stated elsewhere, suitable optional substituents for a particular group are those as stated for similar groups herein.

Suitable pharmaceutically-acceptable salts include acid addition salts such as methanesulfonate, fumarate, hydrochloride, citrate, maleate, tartrate and (less preferably) hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. A preferred pharmaceutically-acceptable salt is the sodium salt.

However, to facilitate isolation of the salt during preparation, salts which are less soluble in the chosen solvent may be preferred whether pharmaceutically-acceptable or not.

The compounds of the invention may be administered in the form of a pro-drug which is broken down in the human or animal body to give a compound of the invention. A prodrug may be used to alter or improve the physical and/or pharmacokinetic profile of the parent compound and can be formed when the parent compound contains a suitable group or substituent which can be derivatised to form a prodrug. Examples of pro-drugs include in-vivo hydrolysable esters of a compound of the invention or a pharmaceutically-acceptable salt thereof. Further examples of pro-drugs include in-vivo hydrolysable amides of a compound of the invention or a pharmaceutically-acceptable salt thereof.

Various forms of prodrugs are known in the art, for examples see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

Suitable pro-drugs for pyridine or triazole derivatives include acyloxymethyl pyridinium or triazolium salts eg halides; for example a pro-drug such as: 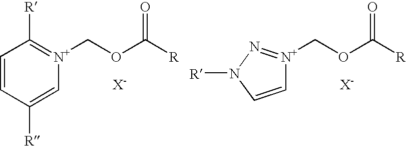
(Ref: T. Yamazaki et al. 42^{nd} Interscience Conference on Antimicrobial Agents and Chemotherapy, San Diego, 2002; Abstract F820).

Suitable pro-drugs of hydroxyl groups are acyl esters of acetal-carbonate esters of formula RCOOC(R,R′)OCO—, where R is (1-4C)alkyl and R′ is (1-4C)alkyl or H. Further suitable prodrugs are carbonate and carbamate esters RCOO— and RNHCOO—.

An in-vivo hydrolysable ester of a compound of the invention or a pharmaceutically-acceptable salt thereof containing a carboxy or hydroxy group is, for example, a pharmaceutically-acceptable ester which is hydrolysed in the human or animal body to produce the parent alcohol.

Suitable pharmaceutically-acceptable esters for carboxy include (1-6C)alkoxymethyl esters for example methoxymethyl, (1-6C)alkanoyloxymethyl esters for example pivaloyloxymethyl, phthalidyl esters, (3-8C)cycloalkoxycarbonyloxy(1-6C)alkyl esters for example 1-cyclohexylcarbonyloxyethyl; 1,3-dioxolan-2-onylnethyl esters for example 5-methyl-1,3-dioxolan-2-ylmethyl; and (1-6C)alkoxycarbonyloxyethyl esters for example 1-methoxycarbonyloxyethyl and may be formed at any carboxy group in the compounds of this invention.

An in-vivo hydrolysable ester of a compound of the invention or a pharmaceutically-acceptable salt thereof containing a hydroxy group or groups includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxymethoxy. A selection of in-vivo hydrolysable ester forming groups for hydroxy include (1-10C)alkanoyl (for example (1-4C)alkanoyl), benzoyl, phenylacetyl and substituted benzoyl and phenylacetyl, (1-10C)alkoxycarbonyl (to give alkyl carbonate esters), di-(1-4C)alkylcarbamoyl and N-(di-(1-4C)alkylaminoethyl)-N-(1-4C)alkylcarbamoyl (to give carbamates), di-(1-4C)alkylaminoacetyl, carboxy(2-5C)alkylcarbonyl and carboxyacetyl. Examples of ring substituents on phenylacetyl and benzoyl include chloromethyl or aminomethyl, (1-4C)alkylaminomethyl and di-((1-4C)alkyl)aminomethyl, and morpholino or piperazino linked from a ring nitrogen atom via a methylene linking group to the 3- or 4-position of the benzoyl ring. Other interesting in-vivo hydrolysable esters include, for example, R^{A}C(O)O(1-6C)alkyl-CO— (wherein R^{A} is for example, optionally substituted benzyloxy-(1-4C)alkyl, or optionally substituted phenyl; suitable substituents on a phenyl group in such esters include, for example, 4-(1-4C)piperazino-(1-4C)alkyl, piperazino-(1-4C)alkyl and morpholino-(1-4C)alkyl.

Further suitable in-vivo hydrolysable esters are those formed from amino acids. For examples, esters formed by reaction of a hydroxy group of a compound with the carboxylic acid of an amino acid. By the term “amino acid” herein we mean any α- or other amino substituted acid, naturally occurring or otherwise ie. non-naturally occurring, and derivatives thereof such as those formed by substitution (for example by alkylation on the nitrogen of the amino group). The use of either a natural or a non-natural amino acid represent particular and independent aspects of the invention. Examples of suitable α-amino acids and derivatives thereof, are valine, leucine, iso-leucine, N-methyl isoleucine, N-tert-butyl-isoleucine, lysine, glycine, N-methylglycine, N,N-dimethyl glycine, alanine, gluamine, asparagine, proline, and phenylalanine. In one embodiment, preferred amino acids are naturally occurring α-amino acids and N-alkylated derivatives thereof.

The use of amino acids having neutral and/or basic side chains represent particular and independent aspects of the invention.

Suitable in-vivo hydrolysable esters of a compound of the formula (I) are described as follows. For example, a 1,2-diol may be cyclised to form a cyclic ester of formula (PD1) or a pyrophosphate of formula (PD2), and a 1,3-diol may be cyclised to form a cyclic ester of the formula (PD3):

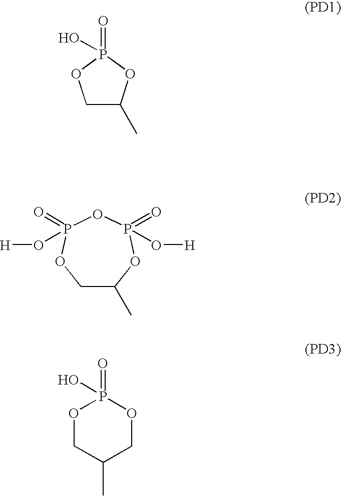

Esters of compounds of formula (I) wherein the HO— function/s in (PD1), (PD2) and (PD3) are protected by (1-4C)alkyl, phenyl or benzyl are useful intermediates for the preparation of such pro-drugs.

Further in-vivo hydrolysable esters include phosphoramidic esters, and also compounds of invention in which any free hydroxy group independently forms a phosphoryl (npd is 1) or phosphiryl (npd is 0) ester of the formula (PD4):

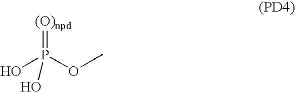

For the avoidance of doubt, phosphono is —P(O)(OH)₂; (1-4C)alkoxy(hydroxy)-phosphoryl is a mono-(1-4C)alkoxy derivative of —O—P(O)(OH)₂; and di-(1-4C)alkoxyphosphoryl is a di-(1-4C)alkoxy derivative of —O—P(O)(OH)₂.

Useful intermediates for the preparation of such esters include compounds containing a group/s of formula (PD4) in which either or both of the —OH groups in (PD1) is independently protected by (1-4C)alkyl (such compounds also being interesting compounds in their own right), phenyl or phenyl-(1-4C)alkyl (such phenyl groups being optionally substituted by 1 or 2 groups independently selected from (1-4C)alkyl, nitro, halo and (1-4C)alkoxy).

Thus, prodrugs containing groups such as (PD1), (PD2), (PD3) and (PD4) may be prepared by reaction of a compound of invention containing suitable hydroxy group/s with a suitably protected phosphorylating agent (for example, containing a chloro or dialkylamino leaving group), followed by oxidation (if necessary) and deprotection.

Other suitable prodrugs include phosphonooxymethyl ethers and their salts, for example a prodrug of R—OH such as:

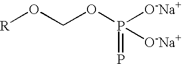

When a compound of invention contains a number of free hydroxy group, those groups not being converted into a prodrug functionality may be protected (for example, using a t-butyl-dimethylsilyl group), and later deprotected. Also, enzymatic methods may be used to selectively phosphorylate or dephosphorylate alcohol functionalities.

Examples of pro-drugs for an amino group include in-vivo hydrolysable amides or a pharmaceutically-acceptable salt thereof. Suitable in-vivo hydrolysable groups include N-carbomethoxy and N-acetyl. Such amides may formed by reaction of an amino (or alkylamino) group with an activated acyl derivative such as an activated ester or an acid chloride, for example, (1-6C)alkanoylchlorides (such as tBuCOCl or acetyl chloride), or substituted derivatives thereof.

A suitable value for an in-vivo hydrolysable amide of a compound of the formula (I) containing a carboxy group is, for example, a N—C₁₋₆alkyl or N,N-di-C₁₋₆alkyl amide such as N-methyl, N-ethyl, N-propyl, N,N-dimethyl, N-ethyl-N-methyl or N,N-diethyl amide. Further suitable values for in-vivo hydrolysable amides of a compound of the formula (I) containing an amine or carboxy group are in-vivo hydrolysable amides formed by reaction with amino-acids, as defined and described herein for in-vivo hydrolysable esters.

Where pharmaceutically-acceptable salts of an in-vivo hydrolysable ester or amide may be formed this is achieved by conventional techniques. Thus, for example, compounds containing a group of formula (PD1), (PD2), (PD3) and/or (PD4) may ionise (partially or fully) to form salts with an appropriate number of counter-ions. Thus, by way of example, if an in-vivo hydrolysable ester prodrug of a compound of invention contains two (PD4) groups, there are four HO—P— functionalities present in the overall molecule, each of which may form an appropriate salt (i.e. the overall molecule may form, for example, a mono-, di-, tri- or tetra-sodium salt).

In one aspect, suitable pro-drugs of the invention are in-vivo hydrolysable esters such as (1-4C)alkyl esters; (1-4C)alkyl esters substituted with (1-4C)alkoxy, (1-4C)alkoxy(1-4C)alkoxy, carboxy, (1-4C)alkyl esters, amino, (1-4C)alkylamino, di(1-4C)alkylamino, tri(1-4C)alkylamino (thereby containing a quaternised nitrogen atom), aminocarbonyl, carbamates, amides or heterocyclyl groups (for example, an ester formed by reaction of a hydroxy group in R⁴ or R⁵ with methoxy acetic acid, methoxypropionic acid, adipic acid momethylester, 4-dimethylaminobutanoic acid, 2-methylaminobutanoic acid, 5-amino pentanoic acid, β-alanine, N,N-diethylalanine, valine, leucine, iso-leucine, N-methyl isoleucine, N-tert-butyl-isoleucine, lysine, glycine, N,N-dimethyl glycine, alanine, sarcosine, glutamine, asparagine, proline, phenylalanine, nicotinic acid, nicotinic acid-N-oxide, pyrimidine-carboxylic acid (for example pyrimidine-5-carboxylic acid), pyrazine-carboxylic acid (for example pyrazine-2-carboxylic acid), or piperidine-4-carboxylic acid); (3-6C)cycloalkyl esters (optionally substituted by a (1-4C)alkoxycarbonyl, alkoxy or carboxy group); carbonates (for example (1-4C)alkylcarbonates and such carbonates substituted by (1-4C)alkoxy or di(1-4C)alkyl)amino); sulfates; phosphates and phosphate esters; and carbamates (see for example Example 10); and pharmaceutically acceptable salts thereof Further suitable pro-drugs are those formed by reaction of a hydroxy group in R⁴ or R⁵ with carbonates, particularly alkoxysubstituted alkyl carbonates such as methoxypropylcarbonate.

Further suitable pro-drugs are esters formed by reaction of a hydroxy group in R⁴ or R⁵ with methoxy acetic acid, methoxypropionic acid, adipic acid momethylester, 4-dimethylaminobutanoic acid, 2-methylaminobutanoic acid, 5-amino pentanoic acid, β-alanine, N,N-diethylalanine, valine, leucine, iso-leucine, N-methyl isoleucine, N-tert-butyl-isoleucine, lysine, glycine, N,N-dimethyl glycine, alanine, sarcosine, glutamine, asparagine, proline, phenylalanine, nicotinic acid, nicotinic acid-N-oxide, pyrimidine-5-carboxylic acid, pyrazine-2-carboxylic acid, or piperidine-4-carboxylic acid, 2-carboxy-cyclohexane-1-carboxylic acid; and pharmaceutically acceptable salts thereof.

Particular compounds of the invention are in-vivo hydrolysable esters formed from amino acids, and pharmaceutically acceptable salts thereof.

Further particular compounds of the invention are in-vivo hydrolysable esters formed from 4-dimethylaminobutanoic acid, 2-methylaminobutanoic acid, 5-amino pentanoic acid, β-alanine, N,N-diethylalanine, valine, leucine, iso-leucine, N-methyl isoleucine, N-tert-butyl-isoleucine, lysine, glycine, N,N-dimethyl glycine, alanine, sarcosine, glutamine, asparagine, proline, phenylalanine; and pharmaceutically acceptable salts thereof.

Further particular compounds of the invention are in-vivo hydrolysable esters formed from valine, leucine, iso-leucine, N-methyl isoleucine, N-tert-butyl-isoleucine, lysine, glycine, N,N-dimethyl glycine, alanine, sarcosine, glutamine, asparagine, proline and phenylalanine; and pharmaceutically acceptable salts thereof.

The compounds of the present invention have a chiral centre at the C-5 positions of the oxazolidinone ring. The pharmaceutically active diastereomer is of the formula (Ia): 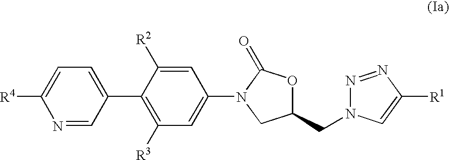
which is the (5R) configuration.

The present invention includes pure diastereomers or mixtures of diastereomers, for example a racemic mixture. If a mixture of enantiomers is used, a larger amount (depending upon the ratio of the enantiomers) will be required to achieve the same effect as the same weight of the pharmaceutically active enantiomer.

Furthermore, some compounds of the invention may have other chiral centres. It is to be understood that the invention encompasses all such optical and diastereoisomers, and racemic mixtures, that possess antibacterial activity. It is well known in the art how to prepare optically-active forms (for example by resolution of the racemic form by recrystallisation techniques, by chiral synthesis, by enzymatic resolution, by biotransformation or by chromatographic separation) and how to determine antibacterial activity as described hereinafter.

The invention relates to all tautomeric forms of the compounds of the invention that possess antibacterial activity.

It is also to be understood that certain compounds of the invention can exist in solvated as well as unsolvated forms such as, for example, hydrated forms. It is to be understood that the invention encompasses all such solvated forms which possess antibacterial activity.

It is also to be understood that certain compounds of the invention may exhibit polymorphism, and that the invention encompasses all such forms which possess antibacterial activity.

As stated before, we have discovered a range of compounds that have good activity against a broad range of Gram-positive pathogens including organisms known to be resistant to most commonly used antibiotics, together with activity against fastidious Gram negative pathogens such as H. influenzae, M. catarrhalis, Mycoplasma and Chlamydia strains. The following compounds possess preferred pharmaceutical and/or physical and/or pharmacokinetic properties.

Whilst we do not want to be bound by theoretical considerations, the inclusion of flexible substituents on the pyridine ring is believed to have a beneficial effect on the solubility of the compounds. For example, in pH 7.4 phosphate buffer, Reference Example 4 as an equilibrium solubility of 35.1 μM and Reference Example 5 has an equilibrium solubility of <7.1 μM. In contrast Example 1 has an equilibrium aqueous solubility of 1453 μM (as the HCl salt). It will be understood that parameters such as solubility may be measured by any suitable technique known in the art.

In one embodiment of the invention are provided compounds of formula (I), in an alternative embodiment are provided pharmaceutically-acceptable salts of compounds of formula (I), in a further alternative embodiment are provided in-vivo hydrolysable esters of compounds of formula (I), and in a further alternative embodiment are provided pharmaceutically-acceptable salts of in-vivo hydrolysable esters of compounds of formula (I). In a further aspect there is provided in-vivo hydrolysable amides of compounds of formula (I).

In one aspect, R¹ is selected from hydrogen, halogen, cyano, methyl, cyanomethyl, fluoromethyl, difluoromethyl, trifluoromethyl, ethynyl and propynyl.

In another aspect, R¹ is selected from hydrogen, chloro, bromo, methyl and fluoromethyl.

In another aspect, R¹ is hydrogen.

In one aspect, R² and R³ are independently hydrogen or fluoro.

In another aspect R² and R³ are both hydrogen.

In another aspect one R² and R³ is hydrogen and the other is fluorine.

In one aspect R⁴ is —(O)R⁵.

In another aspect R⁴ is —C(H)═N—OR⁸. In this aspect, in one embodiment, aspect R⁸ is (1-6C)alkyl (substituted with 1 or 2 substituents independently selected from hydroxy, carboxy, (1-4C)alkoxy and NR⁶R⁷). In another embodiment of this aspect, R⁸ is (3-6C)alkyl (substituted with 1 or 2 substituents independently selected from hydroxy, carboxy, (1-4C)alkoxy and NR⁶R⁷). In another embodiment of this aspect, R⁸ is (1-4C)alkyl (substituted with 1 or 2 substituents independently selected from hydroxy, carboxy, (1-4C)alkoxy and NR⁶ R⁷).

In another aspect R⁴ is —C(R⁵)═N—OH. Embodiments of this aspect comprise any set of values for R⁵ given in any aspect or embodiment herein.

In another aspect R⁴ is —C(R⁵)N—OR⁸. Embodiments of this aspect comprise any set of values for R⁸ given in any aspect or embodiment herein.

In one aspect R⁵ is (1-6C)alkyl (substituted with 1 or 2 substituents independently elected from hydroxy, carboxy, (1-4C)alkoxy, HET-1, and NR⁶R⁷).

In another aspect R⁵ is (3-6C)alkyl (substituted with 1 or 2 substituents independently selected from hydroxy, carboxy, (1-4C)alkoxy, HET-1, and NR⁶R⁷).

In another aspect R⁵ is (1-4C)alkyl (substituted with 1 or 2 substituents independently selected from hydroxy, carboxy, (1-4C)alkoxy, HET-1, and NR⁶R⁷).

In this aspect, R⁵ is preferably not hydroxymethyl.

Suitable values for HET-1 as a substituent on a (1-6C)alkyl group in R⁵ are morpholine, piperazine, N-methylpiperazine, thiomorpholine (and derivatives thereof wherein the sulfur is oxidised to an S(O) or S(O)₂ group), piperidine, pyrrolidine and tetrahydropyridine. A further suitable value for HET-1 as a substituent on a (1-6C)alkyl group in R⁵ is azetidine. In one embodiment, suitable values for HET-1 as a substituent on a (1-6C)alkyl group in R⁵ are piperidine and azetidine.

In another aspect R⁵ is (1-6C)alkyl (substituted with 1 or 2 substituents independently selected from hydroxy, carboxy, (1-4C)alkoxy and NR⁶R⁷).

In another aspect R⁵ is (3-6C)cycloalkyl (optionally substituted with 1 substituent selected from hydroxy, carboxy, (1-4C)alkoxy and NR⁶R⁷);

In another aspect R⁵ is HET-1. Suitable values for R⁵ as HET-1 are morpholine, piperazine, N-methylpiperazine, thiomorpholine (and derivatives thereof wherein the sulfur is oxidised to an S(O) or S(O)₂ group), piperidine, pyrrolidine and tetrahydropyridine. A further suitable value for R⁵ as HET-1 is azetidine. In one embodiment, R⁵ as HET-1 is selected from piperidine and azetidine.

In a further aspect R⁵ is (1-6C)allyl substituted with NR⁶R⁷. In a further aspect R⁵ is (1-6C)alkyl substituted with morpholine, piperazine, N-methylpiperazine, thiomorpholine (or derivatives thereof wherein the sulfur is oxidised to an S(O) or S(O)₂ group), piperidine, pyrrolidine, tetrahydropyridine or imidazole (optionally substituted by 1 or 2 methyl groups). In a further aspect R⁵ is (1-6C)alkyl substituted with morpholine, imidazole, methylimidazole or dimethylimidazole.

In a further aspect, R⁵is (1-6C)alkyl substituted with NR⁶R⁷ wherein NR⁶R⁷ is selected from morpholine, piperazine, N-methylpiperazine, imidazole, methylimidazole, dimethylimidazole, propylimidazole, ethylimidazole and methoxymethylimidazole.

In a further aspect R⁵ is (1-6C)alkyl substituted with morpholine, imidazole, 2-methylimidazole, 4-methylimidazole, 2,5-dimethylimidazole, 2,4-dimethylimidazole, 2-ethylimidazole, 2-n-propylimidazole, 2-isopropylimidazole, or 2-methoxymethylimidazole.

In one aspect, R⁶ and R⁷ are independently selected from hydrogen, methyl, cyclopropyl (optionally substituted with methyl), carboxymethyl and (2-4C)alkyl (optionally substituted by one or two substituents independently selected from amino, (1-4C)alkylamino, di-(1-4C)alkylamino, carboxy, (1-4C)alkoxy and hydroxy).

In another aspect, R⁶ and R⁷ are independently selected from hydrogen, methyl, carboxymethyl and (2-4C)alkyl (optionally substituted by one or two substituents independently selected from amino, (1-4C)alkylamino, di-(1-4C)alkylamino, carboxy, (1-4C)alkoxy and hydroxy).

In another aspect, R⁶ and R⁷ are independently selected from hydrogen and (1-4C)alkyl.

In another aspect, R⁶ and R⁷ are independently selected from hydrogen, methyl, carboxymethyl and (2-4C)alkyl (optionally substituted by one or two substituents independently selected from amino, (1-4C)alkylamino, di-(1-4C)alkylamino, carboxy and hydroxy).

In another aspect, R⁶ and R⁷ are independently selected from hydrogen, methyl, and (2-4C)alkyl (optionally substituted by one or two substituents independently selected from amino, (1-4C)alkylamino, di-(1-4C)alkylamino and hydroxy).

In another aspect, R⁶ and R⁷ are independently selected from hydrogen, methyl, and (2-4C)alkyl (optionally substituted by one or two hydroxy).

In another aspect R⁶ and R⁷ together with a nitrogen to which they are attached form a 4, 5 or 6 membered, saturated or partially unsaturated heterocyclyl ring, optionally containing 1 further heteroatom (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen to which R⁵ and R⁶ are attached is not thereby quaternised) by 1 or 2 (1-4C)alkyl groups.

Suitable values for such a ring comprising R⁶ and R⁷ together with the nitrogen to which they are attached are azetidine, morpholine, piperazine, N-methylpiperazine, thiomorpholine (and derivatives thereof wherein the sulfur is oxidised to an S(O) or S(O)₂ group), piperidine, pyrrolidine and tetrahydropyridine.

In another aspect R⁶ and R⁷ together with a nitrogen to which they are attached form an imidazole, methylimidazole or dimethylimidazole ring, particularly methylimidazole or dimethylimidazole, more particularly dimethylimidazole. In one embodiment, R⁶ and R⁷ together with a nitrogen to which they are attached form 2,4-dimethylimidazole. In another embodiment, R⁶ and R⁷ together with a nitrogen to which they are attached form 2,5-dimethylimidazole.

Further suitable values for such a ring comprising R⁶ and R⁷ together with the nitrogen to which they are attached are morpholine, piperazine, N-methylpiperazine, and thiomorpholine (and derivatives thereof wherein the sulfur is oxidised to an S(O) or S(O)₂ group).

Further suitable values for such a ring comprising R⁶ and R⁷ together with the nitrogen to which they are attached are morpholine and thiomorpholine (and derivatives thereof wherein the sulfur is oxidised to an S(O) or S(O)₂ group).

A particular value is morpholine. A further particular value is imidazole. A further particular value is methylimidazole. A still further particular value is dimethylimidazole.

In one aspect R⁸ is (1-4C)alkyl (optionally substituted with 1 or 2 substituents independently selected from hydroxy, carboxy, (1-4C)alkoxy and NR⁶R⁷).

In a preferred aspect of the invention, the compound of formula (I) is a compound of the formula (Ia).

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro; and

R⁴ is —C(O)R⁵.

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro;

R⁴ is —C(O)R⁵; and

R⁵ is (1-6C)alkyl (substituted with 1 or 2 substituents independently selected from hydroxy, carboxy, (1-4C)alkoxy and NR⁶R⁷).

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro;

R⁴ is —C(O)R⁵;

R⁵ is (1-6C)alkyl (substituted with 1 or 2 substituents independently selected from hydroxy, carboxy, (1-4C)alkoxy and NR⁶R⁷);

R⁶ and R⁷ together with the nitrogen to which they are attached form an imidazole, methylimidazole or dimethylimidazole ring.

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro;

R⁴ is —C(O)R⁵;

R⁵ is (1-6C)alkyl substituted with NR⁶R⁷);

R⁶ and R⁷ together with the nitrogen to which they are attached form an imidazole, methylimidazole, dimethylimidazole, ethylimidazole, propylimidazole or methoxymethylimidazole ring.

In a further aspect of the invention, there is provided a compound of the formula (Ia) as hereinbefore defined, or a pharmaceutically-acceptable salt or prodrug thereof, wherein:

R¹ is selected from hydrogen, chloro, bromo, methyl and fluoromethyl;

R² and R³ are independently hydrogen or fluoro;

R⁴ is —C(O)R⁵;

R⁵ is (1-6C)alkyl substituted with NR⁶R⁷);

R⁶ and R⁷ together with the nitrogen to which they are attached form an imidazole, methylimidazole, dimethylimidazole, ethylimidazole or propylimidazole ring.

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro;

R⁴ is —C(O)R⁵;

R⁵ is (1-6C)alkyl substituted with NR⁶R⁷);

R⁶ and R⁷ together with the nitrogen to which they are attached form an imidazole, methylimidazole, 2,4-dimethylimidazole, 2,5-dimethylimidazole, ethylimidazole or propylimidazole ring.

In a further aspect of the invention, there is provided a compound of the formula (Ia) as hereinbefore defined, or a pharmaceutically-acceptable salt or pro-drug thereof, wherein:

R¹ is hydrogen;

R² and R³ are independently hydrogen or fluoro;

R⁴ is —C(O)R⁵;

R⁵ is (1-6C)alkyl substituted with NR⁶R⁷);

R⁶ and R⁷ together with the nitrogen to which they are attached form an imidazole, methylimidazole, 2,4-dimethylimidazole, 2,5-dimethylimidazole, ethylimidazole or propylimidazole ring.

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro;

R⁴ is —C(O)R⁵; and

R⁵ is HET-1.

In a further aspect of the invention, there is provided a compound of the formula (Ia) as hereinbefore defined, or a pharmaceutically-acceptable salt or pro-drug thereof, wherein:

R¹ is selected from hydrogen, chloro, bromo, methyl and fluoromethyl;

R² and R³ are independently hydrogen or fluoro; and

R⁴ is selected from —(H)═N—OR⁸, —C(R⁵)═N—OH and —C(⁵)═N—OR⁸.

Particular compounds of the present invention include each individual compound described in the Examples, each of which provides an independent aspect of the invention. In another aspect of the invention, there is provided any two or more of the Examples.

Process Section:

In a further aspect the present invention provides a process for preparing a compound of invention or a pharmaceutically-acceptable salt or an in-vivo hydrolysable ester thereof. It will be appreciated that during certain of the following processes certain substituents may require protection to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon. Resins may also be used as a protecting group.

The protecting groups may be removed at any convenient stage in the synthesis using conventional techniques well known in the chemical art.

A compound of the invention, or a pharmaceutically-acceptable salt or an in vivo hydrolysable ester thereof, may be prepared by any process known to be applicable to the preparation of chemically-related compounds. Such processes, when used to prepare a compound of the invention, or a pharmaceutically-acceptable salt or an in vivo hydrolysable ester thereof, are provided as a further feature of the invention and are illustrated by the following representative examples. Necessary starting materials may be obtained by standard procedures of organic chemistry (see, for example, Advanced Organic Chemistry (Wiley-Interscience), Jerry March or Houben-Weyl, Methoden der Organischen Chemie). The preparation of such starting materials is described within the accompanying non-limiting Examples. Alternatively, necessary starting materials are obtainable by analogous procedures to those illustrated which are within the ordinary skill of an organic chemist. Information on the preparation of necessary starting materials or related compounds (which may be adapted to form necessary starting materials) may also be found in the certain Patent Application Publications, the contents of the relevant process sections of which are hereby incorporated herein by reference; for example WO 94/13649; WO 98/54161; WO 99/64416; WO 99/64417; WO 00/21960; WO 01/40222, WO 01/94342; WO 03/022824, JP2003335762 and WO 03/006440.

In particular we refer to our PCT patent applications WO 99/64417 and WO 00/21960 wherein detailed guidance is given on convenient methods for preparing oxazolidinone compounds.

The skilled organic chemist will be able to use and adapt the information contained and referenced within the above references, and accompanying Examples therein and also the Examples herein, to obtain necessary starting materials, and products.

Thus, the present invention also provides that the compounds of the invention and pharmaceutically-acceptable salts and in vivo hydrolysable esters thereof, can be prepared by a process (a) to (m) (wherein each variable is as defined in Formula (I) unless otherwise stated); and thereafter if necessary:
i) removing any protecting groups;
ii) forming a pro-drug (for example an in-vivo hydrolysable ester); and/or
iii) forming a pharmaceutically-acceptable salt;
wherein said processes (a) to (m) are as follows (wherein the variables are as defined above unless otherwise stated):
a) by modifying a substituent in, or introducing a substituent into another compound of the invention by using standard chemistry (see for example, Comprehensive Organic Functional Group Transformations (Pergamon), Katritzky, Meth-Cohn & Rees); for example:
a hydroxy group may be converted into a fluoro group; into an acyloxy group, for instance an acetoxy group; an amino group; a heterocyclyl group linked through nitrogen (optionally substituted on a carbon other than a carbon atom adjacent to the linking nitrogen ring atom), for instance an optionally substituted imidazole-1-yl group; such conversions of the hydroxy group taking place directly (for instance by acylation or Mitsunobu reaction) or through the intermediacy of one or more derivatives (for instance a mesylate or an azide);
an acyloxy group may be converted into a hydroxy group or into the groups that may be obtained from a hydroxy group (either directly or through the intermediacy of a hydroxy group);
an alkyl halogenide group may be converted to a hydroxyl group; an amino group; a thioalkyl group; a heterocyclyl group linked through nitrogen;
a hydroxyl group may be oxidized to a keto group;
b) by reaction of one part of a compound of formula (II) (wherein X is a leaving group useful in palladium [0] coupling, for example chloride, bromide, iodide, trifluoromethylsulfonyloxy, trimethylstannyl, trialkoxysilyl, or a boronic acid residue) with one part of a compound IIa, again with a leaving group X, such that the pyridyl-phenyl bond replaces the phenyl-X and pyridyl-X bonds; such methods are now well known, see for instance S. P. Stanforth, Catalytic Cross-Coupling Reactions in Biaryl Synthesis, Tetrahedron, 54, 1998, 263-303; J. K. Stille, Angew Chem. Int. Ed. Eng., 1986, 25, 509-524; N. Miyaura and A Suzuki, Chem. Rev., 1995, 95, 2457-2483; D. Baranano, G. Mann, and J. F. Hartwig, Current Org. Chem., 1997, 1, 287-305; S. P. Stanforth, Tetrahedron, 54 1998,263-303; P. R. Parry, C. Wang, A. S. Batsanov, M. R. Bryce; and B. Tarbit, J. Org. Chem., 2002, 67, 7541-7543; 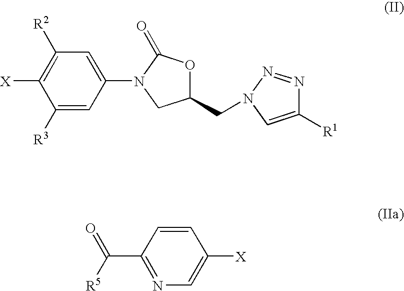
   the leaving group X may be the same or different in the two molecules (II) and (IIa); for example: 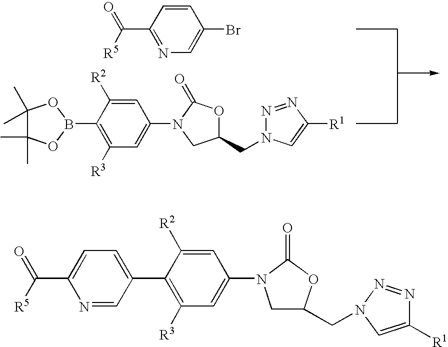
c) by reaction of a pyridyl-phenyl carbamate derivative (III) with an appropriately substituted oxirane to form an oxazolidinone ring; 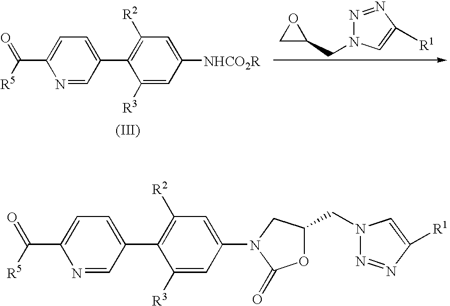
   variations on this process in which the carbamate is replaced by an isocyanate or by an amine or/and in which the oxirane is replaced by an equivalent reagent X—CH₂CH(O-optionally protected)CH₂-triazole R₁ where X is a displaceable group are also well known in the art, for example, 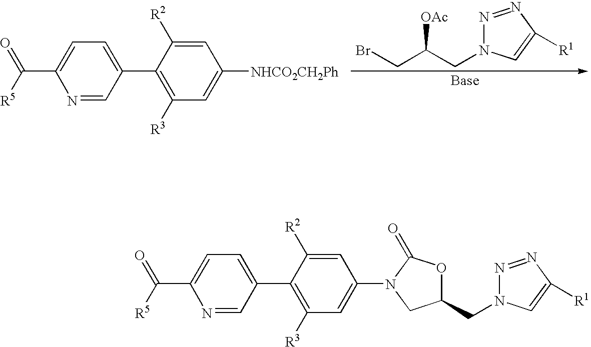
(d) for R⁴ as —COR⁵, by reaction of a compound of formula (IV): 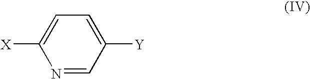
   where X is a replaceable substituent—such as chloride, bromide, iodide, trifluoromethylsulfonyloxy, and 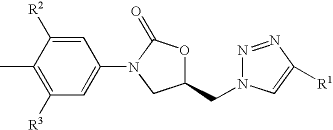
Y is halo or with acylating agents, such as acid chlorides or Weinreb amides, for example: 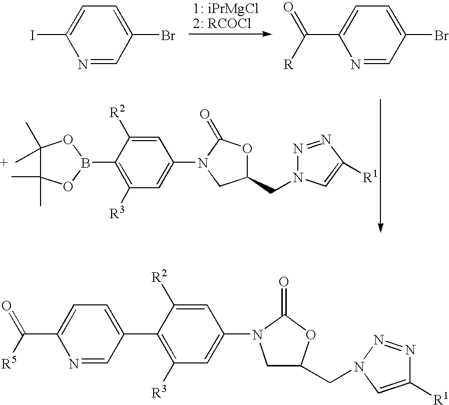
e) for R⁴ as —COR⁵, from an alpha halo ketone derivative by reaction with a nucleophile, to give a compound of formula (IIa), followed by reaction with a compound of formula (II), for example:

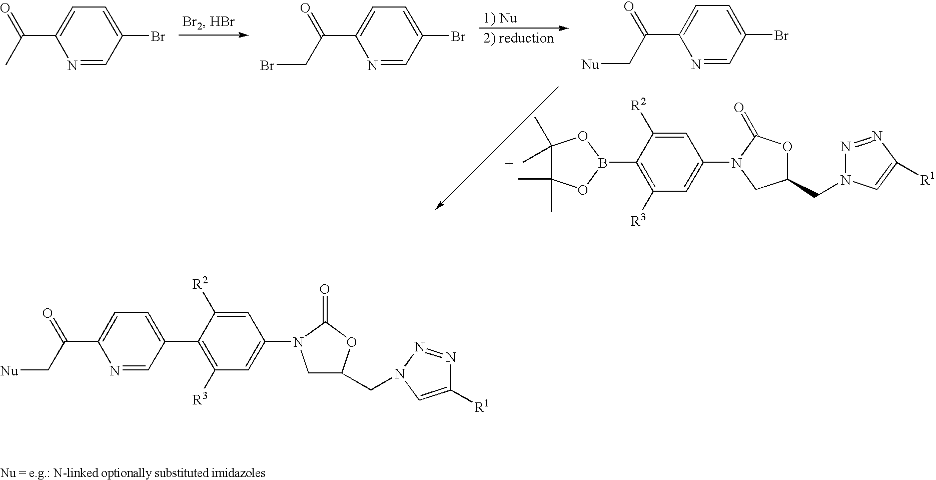

the nucleophile may be protected in order to achieve a desired regiospecificity in the reaction with the alpha halo ketone, followed by a deprotection step thereafter; for example: 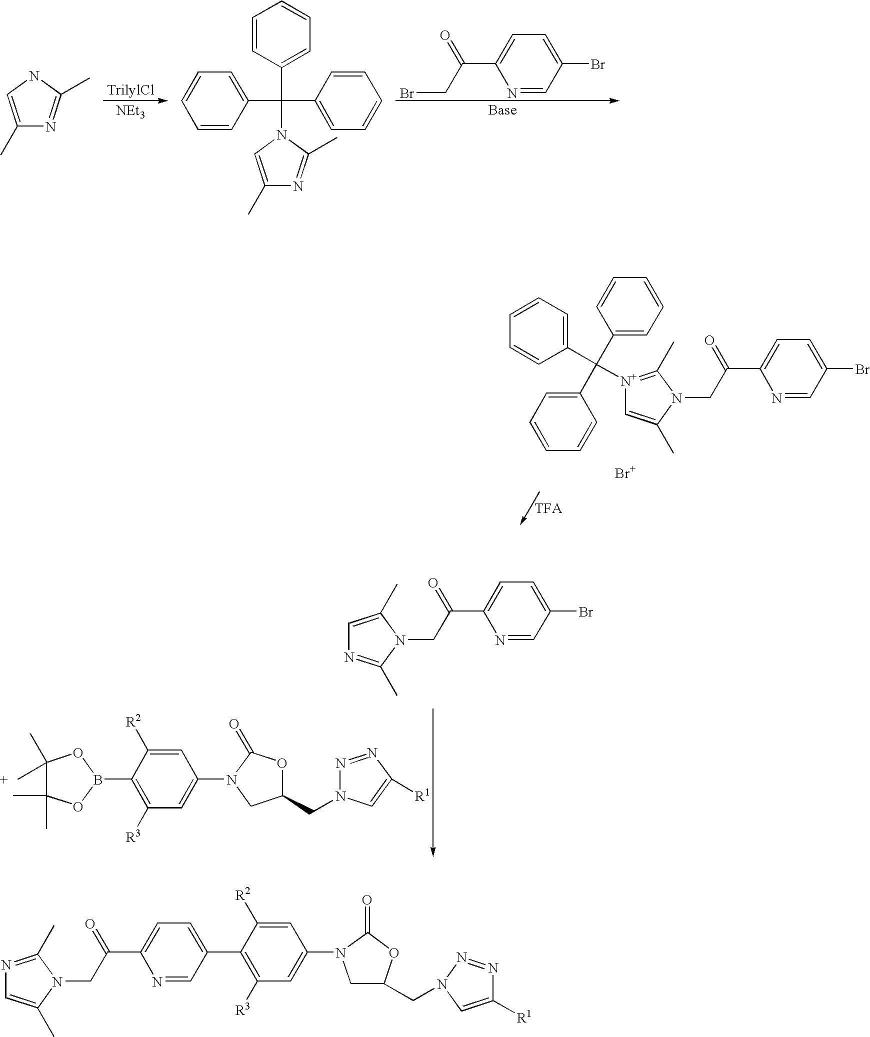
f) for R⁴ as —COR⁵, oxidation of an alcohol derivative; for example: 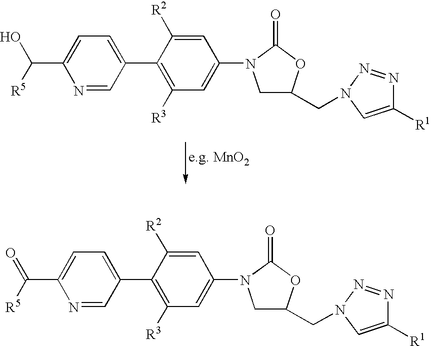
g) for R⁴ as an oxime, by reaction of an aldehyde or ketone with hydroxyl amine or a O-alkylated hydroxyl amine derivative; for example: 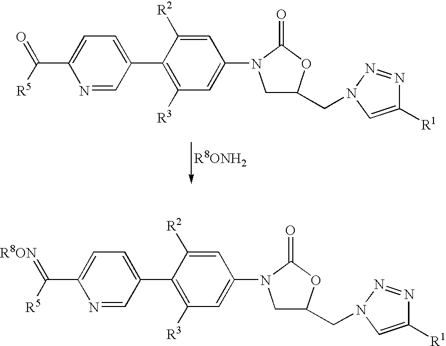
h) for R⁴ as —COR⁵, by reaction of a pyridyl-2-cyano derivative (V) with Grignard Reagents (eg R⁵MgBr) or similar metal alkyl reagents, followed by hydrolysis; 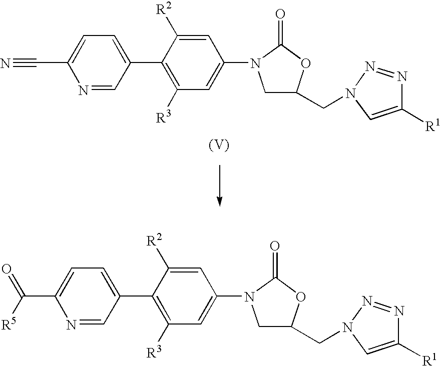
i) for R⁴ as —COR⁵, by alkylation of a pyridyl-2-carboxylate derivative of formula (IV) wherein X is a carboxylate derivative and Y is halo, followed by reaction with a compound of formula (II), for example: 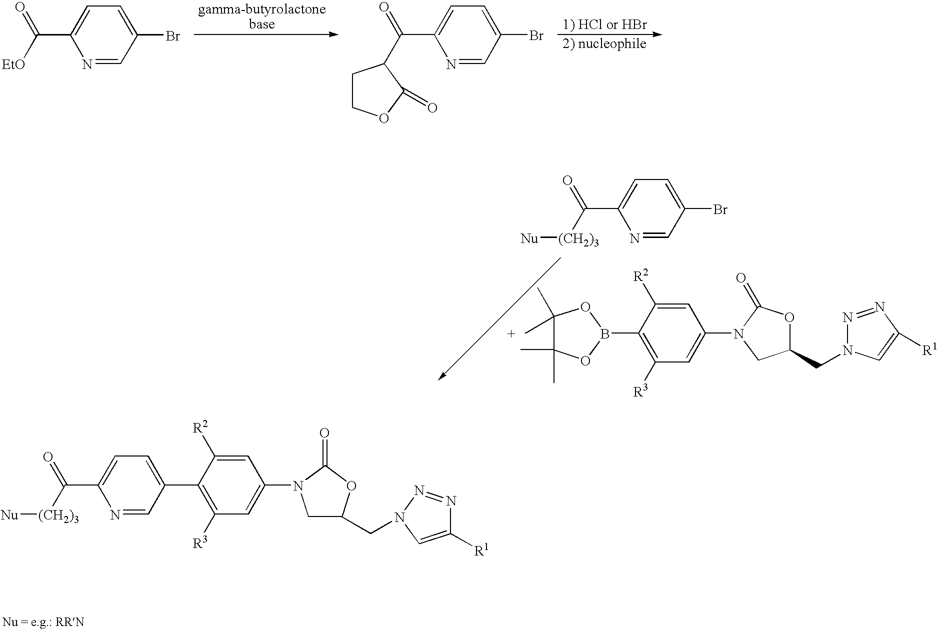
j) by formation of the triazole ring from a suitably functionalised intermediate in which the R⁴-pyridyl-phenyl ring system is already formed, for example as illustrated by the scheme: 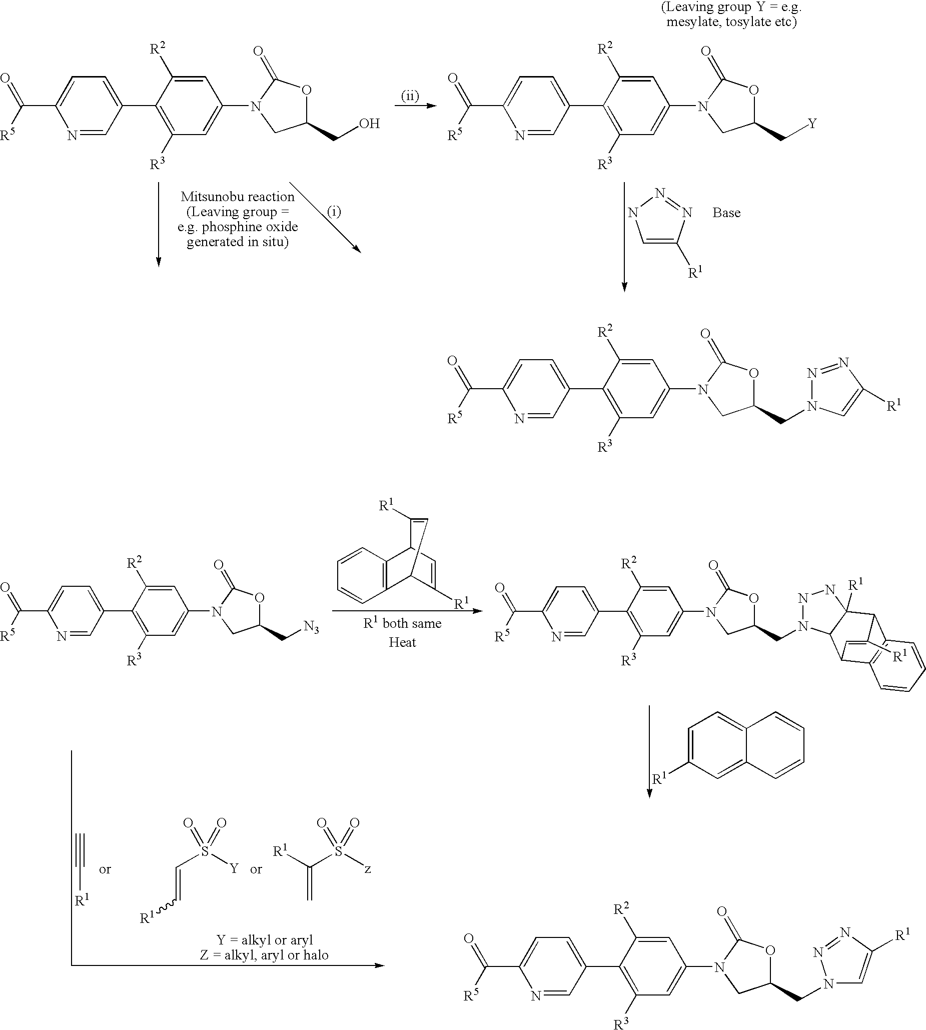
k) by cycloaddition via the azide to acetylenes, for example by reacting azidomethyl oxazolidinones with terminal alkynes using Cu(I) catalysis in e.g. aqueous alcoholic solution at ambient temperatures to give 4-substituted 1,2,3-triazoles (V. V. Rostovtsev, L. G. Green, V. V. Fokin, and K. B. Sharpless, Angew. Chem. Int. Ed., 2002, 41, 2596-2599): 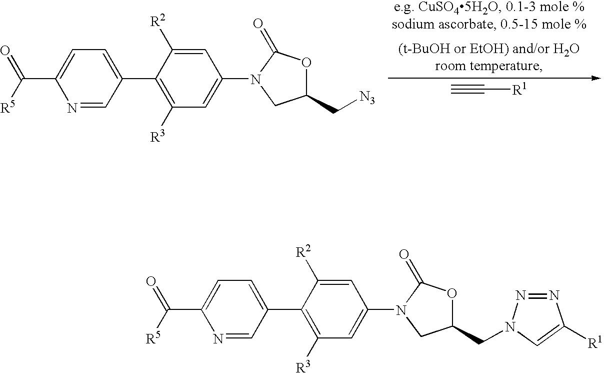
l) by reacting aminomethyloxazolidinones with 1,1-dihaloketone sulfonylhydrazones (Sakai, Kunihazu; Hida, Nobuko; Kondo, Kiyosi; Bull. Chem. Soc. Jpn., 59, 1986, 179-183; Sakai, Kunikazu; Tsunemoto, Daiei; Kobori, Takeo; Kondo, Kiyoshi; Hido, Noboko EP 103840 A2 19840328); 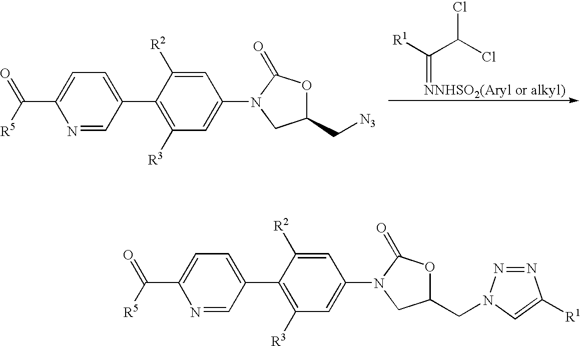
m) for R¹ as 4-halo, compounds of formula (I) may also be made by reacting azidomethyl oxazolidinones with halovinylsulfonyl chlorides at a temperature between 0° C. and 100° C., either without solvent or in an inert diluent such as chloroform or dioxan.

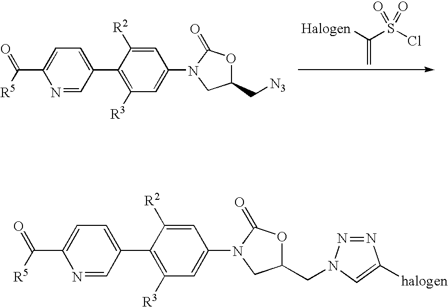

The removal of any protecting groups, the formation of a pharmaceutically-acceptable salt and/or the formation of an in-vivo hydrolysable ester or amide are within the skill of an ordinary organic chemist using standard techniques. Furthermore, details on the these steps, for example the preparation of in-vivo hydrolysable ester prodrugs has been provided, for example, in the section above on such esters.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

Compounds of the formula (II) wherein X is an iodine, tin or boron derivative may be made according to the processes described in WO 03/022824.

Compounds of the formula (II) wherein X═Br (formula (IIc) may be made from compounds of the formula (II) wherein X═H (formula (IIb) by direct bromination of a solution of the compound of formula (IIb) using bromine generated in situ from a bromate, a bromide and an acid (wherein R² and R³ are independently H or F and Rp is selected from hydrogen, halogen, cyano, methyl, cyanomethyl, fluoromethyl, difluoromethyl, trifluoromethyl and —Si[(1-4C)alkyl]₃).

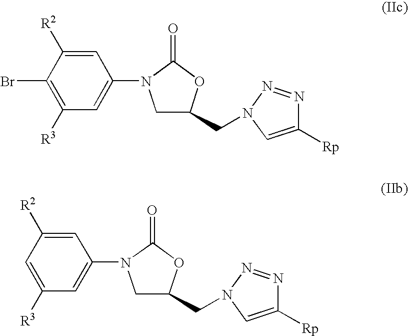

It will be appreciated that producing bromine in the reaction medium, for example by the reaction between a bromate, a bromide and acid, according to the reaction:
BrO₃₋+6H⁺+5Br—→3Br₂+3H₂O
is a convenient way to circumvent problems associated with degradation of bromine solutions with time.

Conveniently, the acid and bromide may be provided together by use of hydrobromic acid. Suitably the bromide is added as a solution in water, for example an aqueous solution of hydrobromic acid, such as a 48% w/w aqueous hydrobromic acid solution. Any convenient concentration of such a solution may be used.

Conveniently the bromate is an alkali metal bromate, such as potassium bromate or sodium bromate. Suitably the bromate is added as a solution in water.

The compound of formula (IIb) may be dissolved in any suitable organic solvent. In this context, suitable means that the organic solvent must be be miscible with water and must not react with the other reagents.

A suitable solvent is acetic acid. The compound of formula (IIb) may be dissolved in a mixture of said suitable organic solvent, such as acetic acid, and water.

Conveniently, the aqueous solution of bromide is added to the solution of the compound of formula (IIb), then the solution of bromate is added.

The reaction between bromate and bromide in the presence of acid is exothermic. Conveniently, a vessel containing the reaction mixture may be cooled, for instance in an ice-bath, but maintenance at a particular temperature is not essential for the yield or quality of the product produced. Conveniently a vessel containing the reaction mixture is cooled in an ice-bath such that the temperature of the reaction ranges between 10 and 30° C. during the addition of bromate.

Suitably slight molar excesses of bromate and bromide are used in comparison to the quantity of the compound of formula (IIb) used.

The rate of addition of the bromate solution is not critical. Conveniently, it is added at a rate such that the temperature of the reaction is maintained between 10 and 30° C. during the addition of bromate.

The reaction mixture may be stirred, for example at about ambient temperature, until the reaction is complete. Typically, the reaction may take 3-4 hours to complete, including the time required for addition of bromate.

After the reaction is complete, it is desirable to remove any excess bromine generated before isolation of the product. Conveniently this may be achieved by addition of a solution of metabisulfite, for example a solution of sodium metabisulfite in water. Sufficient metabisulfite is added to react with any residual bromine.

The product may be isolated by any convenient means, for example by filtration from the reaction mixture, or by dissolution into another organic solvent and appropriate washing and evaporation. If the product solidifies from the reaction mixture, it may be convenient to re-dissolve it (for example by heating the solution, for example to about 80-85° C.) and allow crystallisation in a controlled manner.

According to a further aspect of the invention, there is provided a process for forming a compound of the formula (IIc) from a compound of the formula (IIb) as hereinbefore defined, said process comprising treatment of a solution of the compound of formula (IIb) with an alkali metal bromate, and hydrobromic acid.

According to a further aspect of the invention, there is provided a process for forming a compound of the formula (IIc) from a compound of the formula (IIb) as hereinbefore defined, said process comprising:
a) treatment of a solution of the compound of formula (IIb) in a mixture of water and a suitable organic solvent with aqueous hydrobromic acid; and
b) addition of an aqueous solution of an alkali metal bromate.

According to a further aspect of the invention, there is provided a process for forming a compound of the formula (IIc) from a compound of the formula (IIb) as hereinbefore defined, said process comprising:
a) treatment of a solution of the compound of formula (IIb) in a mixture of water and a suitable organic solvent with aqueous hydrobromic acid;
b) addition of an aqueous solution of an alkali metal bromate; and
c) addition of a solution of sodium metabisulfite to react with any excess bromine.

According to a further aspect of the invention, there is provided a process for forming a compound of the formula (IIc) from a compound of the formula (IIb) as hereinbefore defined, said process comprising:
a) treatment of a solution of the compound of formula (IIb) in a mixture of water and a suitable organic solvent with aqueous hydrobromic acid;
b) addition of an aqueous solution of an alkali metal bromate;
c) addition of a solution of sodium metabisulfite to react with any excess bromine;
d) isolation of the product compound of the formula (IIc).

According to a further aspect of the invention, there is provided a process for forming a compound of the formula (IIc) from a compound of the formula (IIb) as hereinbefore defined, said process comprising:
a) treatment of a solution of the compound of formula (IIb) in a mixture of water and a suitable organic solvent with aqueous hydrobromic acid;
b) addition of an aqueous solution of an alkali metal bromate;
c) addition of a solution of sodium metabisulfite to react with any excess bromine;
d) isolation of the product compound of the formula (IIc) by heating the mixture resulting from step c) until any solid has dissolved and then cooling the solution until the compound of the formula (IIc) crystallises.

According to a further feature of the invention there is provided a compound of the invention, or a pharmaceutically-acceptable salt, or in-vivo hydrolysable ester thereof for use in a method of treatment of the human or animal body by therapy.

According to a further feature of the present invention there is provided a method for producing an antibacterial effect in a warm blooded animal, such as man, in need of such treatment, which comprises administering to said animal an effective amount of a compound of the present invention, or a pharmaceutically-acceptable salt, or in-vivo hydrolysable ester thereof.

The invention also provides a compound of the invention, or a pharmaceutically-acceptable salt, or in-vivo hydrolysable ester thereof, for use as a medicament; and the use of a compound of the invention of the present invention, or a pharmaceutically-acceptable salt, or in-vivo hydrolysable ester thereof, in the manufacture of a medicament for use in the production of an antibacterial effect in a warm blooded animal, such as man.

In order to use a compound of the invention, an in-vivo hydrolysable ester or a pharmaceutically-acceptable salt thereof, including a pharmaceutically-acceptable salt of an in-vivo hydrolysable ester, (hereinafter in this section relating to pharmaceutical composition “a compound of this invention”) for the therapeutic (including prophylactic) treatment of mammals including humans, in particular in treating infection, it is normally formulated in accordance with standard pharmaceutical practice as a pharmaceutical composition.

Therefore in another aspect the present invention provides a pharmaceutical composition which comprises a compound of the invention, an in-vivo hydrolysable ester or a pharmaceutically-acceptable salt thereof, including a pharmaceutically-acceptable salt of an in-vivo hydrolysable ester, and a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration as eye-drops, for administration by inhalation (for example as a finely divided powder or a liquid aerosol), for administration by insufflation (for example as a finely divided powder) or for parenteral administration (for example as a sterile aqueous or oily solution for intravenous, subcutaneous, sub-lingual, intramuscular or intramuscular dosing or as a suppository for rectal dosing).

In addition to the compounds of the present invention, the pharmaceutical composition f this invention may also contain (ie through co-formulation) or be co-administered (simultaneously, sequentially or separately) with one or more known drugs selected from other clinically useful antibacterial agents (for example, β-lactams, macrolides, quinolones or aminoglycosides) and/or other anti-infective agents (for example, an antifungal triazole or amphotericin). These may include carbapenems, for example meropenem or imipenem, to broaden the therapeutic effectiveness. Compounds of this invention may also be co-formulated or co-administered with bactericidal/permeability-increasing protein (BPI) products or efflux pump inhibitors to improve activity against gram negative bacteria and bacteria resistant to antimicrobial agents. Compounds of this invention may also be co-formulated or co-administered with a vitamin, for example Vitamin B, such as Vitamin B2, Vitamin B6, Vitamin B12 and folic acid. Compounds of the invention may also be formulated or co-administered with cyclooxygenase (COX) inhibitors, particularly COX-2 inhibitors.

In one aspect of the invention, a compound of the invention is co-formulated with an antibacterial agent which is active against gram-positive bacteria.

In another aspect of the invention, a compound of the invention is co-formulated with an antibacterial agent which is active against gram-negative bacteria.

In another aspect of the invention, a compound of the invention is co-administered with an antibacterial agent which is active against gram-positive bacteria.

In another aspect of the invention, a compound of the invention is co-administered with an antibacterial agent which is active against gram-negative bacteria.

The compositions of the invention may be obtained by conventional procedures using conventional pharmaceutical excipients, well known in the art. Thus, compositions intended for oral use may contain, for example, one or more colouring, sweetening, flavouring and/or preservative agents. A pharmaceutical composition to be dosed intravenously may contain advantageously (for example to enhance stability) a suitable bactericide, antioxidant or reducing agent, or a suitable sequestering agent.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol. Solubility enhancing agents, for example cyclodextrins may be used.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 50 mg to 5 g of active agent compounded with an appropriate and convenient amount of excipients which may vary from about 5 to about 98 percent by weight of the total composition. Dosage unit forms will generally contain about 200 mg to about 2 g of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

A suitable pharmaceutical composition of this invention is one suitable for oral administration in unit dosage form, for example a tablet or capsule which contains between 1 mg and 1 g of a compound of this invention, preferably between 100 mg and 1 g of a compound. Especially preferred is a tablet or capsule which contains between 50 mg and 800 mg of a compound of this invention, particularly in the range 100 mg to 500 mg.

In another aspect a pharmaceutical composition of the invention is one suitable for intravenous, subcutaneous or intramuscular injection, for example an injection which contains between 0.1% w/v and 50% w/v (between 1 mg/ml and 500 mg/ml) of a compound of this invention.

Each patient may receive, for example, a daily intravenous, subcutaneous or intramuscular dose of 0.5 mgkg⁻¹ to 20 mgkg⁻¹ of a compound of this invention, the composition being administered 1 to 4 times per day. In another embodiment a daily dose of 5 mgkg⁻¹ to 20 mgkg⁻¹ of a compound of this invention is administered. The intravenous, subcutaneous and intramuscular dose may be given by means of a bolus injection. Alternatively the intravenous dose may be given by continuous infusion over a period of time. Alternatively each patient may receive a daily oral dose which may be approximately equivalent to the daily parenteral dose, the composition being administered 1 to 4 times per day.

In the above other, pharmaceutical composition, process, method, use and medicament manufacture features, the alternative and preferred embodiments of the compounds of the invention described herein also apply.

Antibacterial Activity:

The pharmaceutically-acceptable compounds of the present invention are useful antibacterial agents having a good spectrum of activity in vitro against standard Gram-positive organisms, which are used to screen for activity against pathogenic bacteria. Notably, the pharmaceutically-acceptable compounds of the present invention show activity against enterococci, pneumococci and methicillin resistant strains of S. aureus and coagulase negative staphylococci, together with haemophilus and moraxella strains. The antibacterial spectrum and potency of a particular compound may be determined in a standard test system.

The (antibacterial) properties of the compounds of the invention may also be demonstrated and assessed in-vivo in conventional tests, for example by oral and/or intravenous dosing of a compound to a warm-blooded mammal using standard techniques.

The following results were obtained on a standard in-vitro test system. The activity is described in terms of the minimum inhibitory concentration (MIC) determined by the agar-dilution technique with an inoculum size of 10⁴ CFU/spot. Typically, compounds are active in the range 0.01 to 256 μg/ml.

Staphylococci were tested on agar, using an inoculum of 10⁴ CFU/spot and an incubation temperature of 37° C. for 24 hours—standard test conditions for the expression of methicillin resistance.

Streptococci and enterococci were tested on agar supplemented with 5% defibrinated horse blood, an inoculum of 10⁴ CFU/spot and an incubation temperature of 37° C. in an atmosphere of 5% carbon dioxide for 48 hours—blood is required for the growth of some of the test organisms. Fastidious Gram negative organisms were tested in Mueller-Hinton broth, supplemented with hemin and NAD, grown aerobically for 24 hours at 37° C., and with an innoculum of 5×10⁴ CFU/well.

For example, the following results were obtained for the compound of Example 1:

| Organism | | MIC (μg/ml) |
|---|---|---|
| Staphylococcus aureus: | MSQS | 0.25 |
| | MRQR | 0.25 |
| Streptococcus pneumoniae | | 0.13 |
| Haemophilus influenzae | | 2 |
| Moraxella catarrhalis | | 0.5 |
| Linezolid Resistant Streptococcus pneumoniae | | 0.5 |

MSQS = methicillin sensitive and quinolone sensitive
MRQR = methicillin resistant and quinolone resistant

Inhibition of mono-amine oxidase (MAO) is a known potential side effect of oxazolidinone antibiotics (see for example WO 03/072575). The compounds of this invention generally have lower levels of MAO-A inhibition than those exhibited by unsubstituted and other simple substituted pyridine compounds, as shown in the table below.

| Reference | Structure | MAO-A Ki (μM) |
|---|---|---|
| Ref Example 1 | 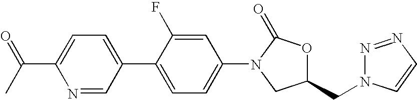 | 0.72 |
| Ref Example 2 | 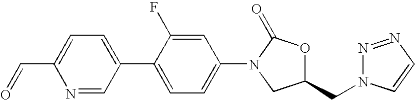 | 0.7 |
| Ref Example 3 | 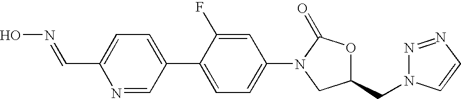 | <0.3 |
| Example 1 | 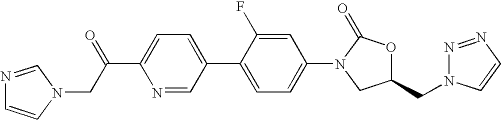 | >5 |
| Example 3 | 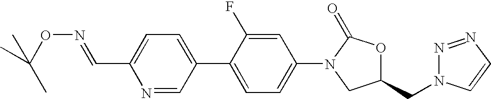 | 6.4 |

The activity of the compounds of the invention against MAO-A was tested using a standard in-vitro assay based on human liver enzyme expressed in yeast as described in Biochem. Biophys. Res. Commun. 1991, 181, 1084-1088. Compounds of the invention typically give Ki values of >5 μM when measured in such an assay as above. Example 1 showed a Ki value of >5 μM and Example 2 showed a Ki value of 15 μM.

It will be appreciated that, as described in our patent application WO 03/072575, compounds with 4-alkyl triazoles generally demonstrate lower MAO-A inhibition than the analogous unsubstituted triazole compounds.

Certain intermediates and/or Reference Examples described hereinafter are within the scope of the invention and may also possess useful activity, and are provided as a further feature of the invention. Particular Reference Examples are Reference Examples 1, 2 and 3.

The invention is now illustrated but not limited by the following Examples in which unless otherwise stated:—
(i) evaporations were carried out by rotary evaporation in-vacuo and work-up procedures were carried out after removal of residual solids by filtration;
(ii) operations were carried out at ambient temperature, that is typically in the range 18-26° C. and without exclusion of air unless otherwise stated, or unless the skilled person would otherwise work under an inert atmosphere;
(iii) column chromatography was used to purify compounds, either by the flash procedure on normal phase silica gel 60, 230-400 mesh, or by the flash procedure on reverse phase silica gel (C-18, RediSep, Isco, Inc.), or by HPLC on reverse phase silica gel (e.g.: Waters YMC-ODS AQ, C-18) using a Gilson 215 Platform, unless otherwise stated;
(iv) yields are given for illustration only and are not necessarily the maximum attainable;
(v) the structure of the end-products of the invention were generally confirmed by NMR and mass spectral techniques [proton magnetic resonance spectra were generally determined in DMSO-d₆ unless otherwise stated, using a Bruker spectrometer at 300, 400 or 500 MHz; chemical shifts are reported in parts per million downfield from tetramethysilane as an internal standard (δ scale) or relative to solvent. Peak multiplicities are shown thus: s, singlet; d, doublet; AB or dd, doublet of doublets; dt, doublet of triplets; dm, doublet of multiplets; t, triplet, m, multiplet; br, broad; mass spectroscopy was performed using a Micromass Quattro Micro mass spectrometer (for ESP) and an Agilent 1100 MSD instrument (for APCI); optical rotations were determined at 589 nm at 20° C. using a Perkin Elmer Polarimeter 341;
(vi) each intermediate was purified to the standard required for the subsequent stage and was characterised in sufficient detail to confirm that the assigned structure was correct; purity was assessed by HPLC, LC-MS, TLC, or NMR and identity was determined by mass spectroscopy and/or NMR spectroscopy as appropriate;
(vii) in which the following abbreviations may be used:—

DMF is N,N-dimethylformamide; DMA is N,N-dimethylacetamide; TLC is thin layer chromatography; HPLC is high pressure liquid chromatography; NMP is N-methylpyrrolidone; DMSO is dimethylsulfoxide; CDCl₃ is deuterated chloroform; MS is mass spectroscopy; ESP is electrospray; EI is electron impact; CI is chemical ionisation; APCI is atmospheric pressure chemical ionisation; EtOAc is ethyl acetate; MeOH is methanol; phosphoryl is (HO)₂—P(O)—O—; phosphiryl is (HO)₂—P—O—; THF is tetrahydrofuran; ether is diethylether; THF is tetrahydrofuran; TFA is trifluoroacetic acid;
viii) temperatures are quoted as ° C.

### EXAMPLE 1

### (5R)-3-{3-Fluoro-4-[6-(1H-imidazol-1-ylacetyl)pyridin-3-yl]phenyl}-5-(1H-1,2,3-triazol-1-ylmethyl)-1,3-oxazolidin-2-one

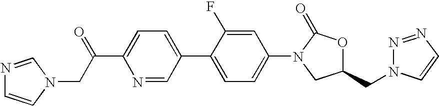

1-(5-Bromopyridin-2-yl)-2-(1H-imidazol-1-yl)ethanone (Intermediate 8) (195 mg, 0.73 mmol), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7,284 mg, 0.73 mmol) and sodium carbonate (233 mg, 2.2 mmol) were dissolved/suspended in DMF/water (5 mL, 10:1). It was degassed, flushed with nitrogen and tetrakis (triphenylphospine) palladium (0) (85 mg, 0.07 mmol) was added. The reaction mixture was heated at 75° C. for 3 hours, cooled to room temperature, and the solvent was evaporated. The residue was purified by chromatography on silica gel with dichloromethane/methanol (10:1). The free base thus obtained was dissolved in isopropanol/dichloromethane (20 mL, 1:1), HCl in ether (1 mL, 1M) was added and most of the dichloromethane was removed under reduced pressure. The residue was collected by filtration and dried to give 189 mg (59%) of product as the hydrochloride salt, colourless solid, mp >230° C. (dec.).

MS (ESP): 448.15 (MH⁺) for C₂₂H₁₈FN₇O₃

¹H-NMR (DMSO-d₆) δ: 3.97 (m, 1H); 4.31 (m, 1H); 4.86 (d, 2H); 5.20 (m, 1H); 6.12 (s, 2H); 7.45 (dd, 1H); 7.62 (dd, 1H); 7.70-7.82 (m, 5H); 8.11-8.20 (m, 2H); 8.29 (m, 1H); 9.05 (d, 1H); 14.62 (brs, 1H).

The intermediates for Example 1 were prepared as follows:

### Intermediate 1: Acetic acid (5R)-3-(3-fluoro-phenyl)-2-oxo-oxazolidin-5-ylmethyl ester

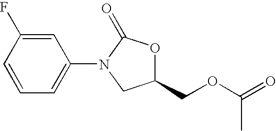

(5R)-3-(3-Fluorophenyl)-5-hydroxymethyloxazolidin-2-one (40 g, 0.189 mol, see Upjohn WO 94-13649) was suspended by stirring in dry dichloromethane (400 mL) under nitrogen. Triethylamine (21 g, 0.208 mol) and 4-dimethylaminopyridine (0.6 g, 4.9 mmol) were added, followed by dropwise addition of acetic anhydride (20.3 g, 0.199 mol) over 30 minutes, and stirring continued at ambient temperature for 18 hours. Saturated aqueous sodium bicarbonate (250 mL) was added, the organic phase separated, washed with 2% sodium dihydrogen phosphate, dried (magnesium sulfate), filtered and evaporated to give the desired product (49.6 g) as an oil.

MS (ESP): 254 (MH⁺) for C₁₂H₁₂FINO₄

NMR(300 MHz) (CDCl₃) δ: 2.02 (s, 3H); 3.84 (dd, 1H); 4.16 (t, 1H); 4.25 (dd, 1H); 4.32 (dd, 1H); 4.95 (m, 1H); 6.95 (td, 1H); 7.32 (d, 1H); 7.43 (t, 1H); 7.51 (d, 1H).

### Intermediate 2: Acetic acid (5R)-3-(3-fluoro-4-iodo-phenyl)-2-oxo-oxazolidin-5-ylmethyl ester

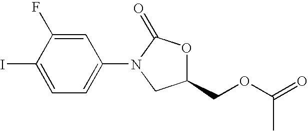

Acetic acid (5R)-3-(3-fluoro-phenyl)-2-oxo-oxazolidin-5-ylmethyl ester (Intermediate 1, 15.2 g, 60 mmol) was dissolved in a mixture of chloroform (100 mL) and acetonitrile (100 mL) under nitrogen, and silver trifluoroacetate (16.96 g, 77 mmol) were added. Iodine (18.07 g, 71 mmol) was added in portions over 30 minutes to the vigorously stirred solution, and stirring continued at ambient temperature for 18 hours. As reaction was not complete, a further portion of silver trifluoroacetate (2.64 g, 12 mmol) was added and stirring continued for 18 hours. After filtration, the mixture was added to sodium thiosulfate solution (3%, 200 mL) and dichloromethane (200 mL), and the organic phase separated, washed with sodium thiosulfate (200 mL), saturated aqueous sodium bicarbonate (200 mL), brine (200 mL), dried (magnesium sulfate), filtered and evaporated. The crude product was suspended in isohexane (100 mL), and sufficient diethyl ether added to dissolve out the brown impurity while stirring for 1 hour. Filtration gave the desired product (24.3 g) as a cream solid.

MS (ESP): 380 (MH⁺) for C₁₂H₁₁FINO₄

NMR(300 MHz) (DMSO-d₆) δ: 2.03 (s, 3H); 3.82 (dd, 1H); 4.15 (t, 1H); 4.24 (dd, 1H); 4.30 (dd, 1H); 4.94 (m, 1H); 7.19 (dd, 1H); 7.55 (dd, 1H); 7.84 (t, 1H).

### Intermediate 3: (5R)-3-(3-Fluoro-4-iodophenyl)-5-hydroxymethyloxazolidin-2-one

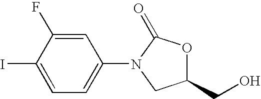

Acetic acid (5R)-3-(3-fluoro-4-iodophenyl)-2-oxo-oxazolidin-5-ylmethyl ester (Intermediate 2, 30 g, 79 mmol) was treated with potassium carbonate (16.4 g, 0.119 mmol) in a mixture of methanol (800 mL) and dichloromethane (240 mL) at ambient temperature for 25 minutes, then immediately neutralised by the addition of acetic acid (10 mL) and water (500 mL). The precipitate was filtered, washed with water, and dissolved in dichloromethane (1.2 L), the solution washed with saturated sodium bicarbonate, and dried (magnesium sulfate). Filtration and evaporation gave the desired product (23 g).

MS (ESP): 338 (MH⁺) for C₁₀H₉FINO₃

NMR (300 MHz)(DMSO-d₆) δ: 3.53 (m, 1H); 3.67 (m, 1H); 3.82 (dd, 1H); 4.07 (t, 1H); 4.70 (m, 1H); 5.20 (t, 1H); 7.21 (dd, 1H); 7.57 (dd, 1H); 7.81 (t, 1H).

### Intermediate 4: [(5R)-3-(3-Fluoro4-iodotphenyl)-2-oxo-1,3-oxazolidin-5-yl]methyl methanesulfonate

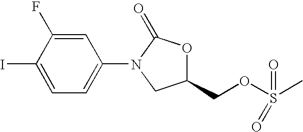

(5R)-3-(3-Fluoro4-iodophenyl)-5-(hydroxymethyl)-1,3-oxazolidin-2-one (Intermediate 3, 25.0 g, 74.2 mmol) was stirred in dichloromethane (250 mL) at 0° C. Triethylamine (10.5 g, 104 mmol) was added followed by methanesulfonyl chloride (11.2 g, 89.0 mmol) and the reaction was stirred overnight, slowly warming to room temperature. The yellow solution was diluted with sodium bicarbonate and the compound was extracted using dichloromethane (3×250 mL). The organic layer was dried (magnesium sulfate), filtered and concentrated to give the desired product as a light yellow solid (30.3 g).

MS (ESP): 416 (MH⁺) for C₁₁H₁₁FINO₅S

¹H-NMR(300 MHz) (DMSO-d₆): 3.24 (s, 3H); 3.82 (dd, 1H); 4.17 (t, 1H); 4.43-4.52 (m, 2H); 4.99-5.03 (m, 1H); 7.21 (dd, 1H); 7.55 (dd, 1H); 7.83 (t, 1H).

### Intermediate 5: (5R)-5-(Azidomethyl)-3-(3-fluoro-4-iodophenyl)-1,3-oxazolidin-2-one

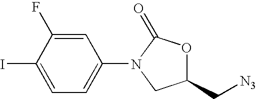

[(5R)-3-(3-Fluoro-4-iodophenyl)-2-oxo-1,3-oxazolidin-5-yl]methyl methanesulfonate (Intermediate 4, 6.14 g, 14.7 mmol) was dissolved in DMF (50 mL). Sodium azide (1.92 g, 29.6 mmol) was added and the reaction was stirred at 75° C. overnight. The yellow mixture was poured into half-saturated sodium bicarbonate and extracted using ethyl acetate. The organic layer was washed three times with water, dried (magnesium sulfate), filtered, and concentrated to give the title compound as a yellow solid (4.72 g).

MS (ESP): 363 (MH⁺) for C₁₀H₈FIN₄O₂

¹H-NMR(300 MHz) (DMSO-d₆): 3.72-3.82 (m, 3H); 4.14 (t, 1H); 4.89-4.94 (m, 1H); 7.22 (dd, 1H); 7.57 (dd, 1H); 7.83 (t, 1H).

### Intermediate 6: (5R)-3-(3-Fluoro-4-iodophenyl)-5-(1H-1,2,3-triazol-1-ylmethyl)-1,3-oxazolidin-2-one

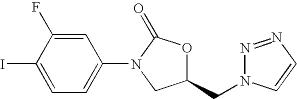

(5R)-5-(Azidomethyl)-3-(3-fluoro-4-iodophenyl)-1,3-oxazolidin-2-one (Intermediate 5, 30.3 g, 72.9 mmol) was stirred in 1,4-dioxane. Bicyclo[2.2.1]hepta-2,5-diene (40.3 g, 437 mmol) was added and the reaction was heated at 100° C. overnight. The resulting brown mixture was filtered and the desired product was obtained as a light brown solid (14.8 g).

MS (ESP): 389 (MH⁺) for C₁₂H₁₀FIN₄O₂

¹H-NMR(300 Mz) (DMSO-d₆: 3.90 (dd, 1H); 4.23 (t, 1H); 4.84 (d, 2H); 5.11-5.18 (m, 1H), 7.14 (dd, 1H); 7.49 (dd, 1H); 7.76 (s, 1H); 7.82 (t, 1H); 8.17 (s, 1H).

### Intermediate 7: (5R)-3-[3-Fluoro-4-(4,4,5,5-tetramethyl-1,3,2-dioxaborolan-2-yl)phenyl]-5-(1H-1,2,3-triazol-1-ylmethyl)-1,3-oxazolidin-2-one

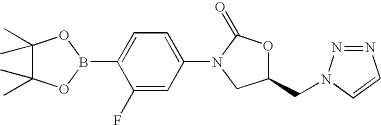

(5R)-3-(3-Fluoro-4-iodophenyl)-5-(1H-1,2,3-triazol-1-ylmethyl)-1,3-oxazolidin-2-one (Intermediate 6, 2 g, 5.15 mmol), bis(pinacolato)diboron, 2.62 g (10.3 mmol), potassium acetate, 2.5 g (25.5 mmol), and 1,1′-[bis(diphenylphosphino)ferrocene]dichloropalladium(II) dichoromethane complex, 0.38 g (0.52 mmol) were suspended in DMSO (15 mL). The mixture was heated at 80° C. for 40 minutes to give a clear black solution. Ethyl acetate (150 mL1) was then added and the mixture was filtered through celite, washed with saturated brine (2×100 mL), dried over sodium sulfate and evaporated. The dark residue was purified by chromatography (silica gel, 40 to 100% ethyl acetate in hexane, followed by 1-5% acetonitrile in ethyl acetate) to give the product as a crystalline tan solid, 1.97 g (98%). (note—highly colored impurities elute ahead of product band, extended elution required to obtain product).

NMR(300 Mz) (DMSO-d₆) δ: 1.28 (s, 12H), 3.91 (dd, 1H); 4.23 (t, 1H); 4.83 (d, 2H); 5.14 (m, 1H); 7.27 (dd, 1H); 7.37 (dd, 1H); 7.62 (t, 1H); 7.75 (s, 1H); 8.16 (s, 1H).

Alternatively:

(5R)-3-(3-Fluoro-4-iodophenyl)-5-(1H-1,2,3-triazol-1-ylmethyl)-1,3-oxazolidin-2-one (Intermediate 6, 5 g, 12.9 mmol), pinacolborane, 2.9 ml (20 mmol), triethylamine, 5.4 ml (39 mmol), and trans-dichlorobis(triphenylphosphine)palladium (II), 0.92 g (1.3 mmol) were dissolved in dioxane (70 mL). The mixture was heated at 100° C. for 90 minutes to give a black solution, which was concentrated, dissolved in ethyl acetate, washed with brine, dried over sodium sulfate and evaporated. The residue was purified by chromatography (silica gel, 0 to 5% methanol in dichloromethane with 1% triethylamine) to give the product (3.1 g) as a light brown solid.

### Intermediate 8: 1-(5-Bromopyridin-2-yl)-2-(1H-imidazol-1-yl)ethanone

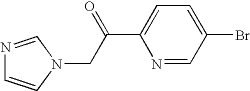

2-Bromo-1-(5-bromopyridin-2-yl)ethanone hydrobromide (Intermediate 9, 440 mg, 1.22 mmol) was suspended in dry THF (5 mL) and cooled to 0° C. Imidazole (330 mg, 4.85 mmol) was added and it was vigorously stirred for 1 hour. The reaction mixture was diluted with dichloromethane, washed with water, and dried over sodium sulfate. Chromatography on silica gel with dichloromethane/methanol (15:1) gave 197 mg of the product (61%), as an off-white solid.

¹H-NMR (MSO-d₆) δ: 5.77 (s, 2H); 6.91 (brs, 1H); 7.13 (brs, 1H); 7.59 (s, 1H); 7.93 (dd, 1H); 8.34 (dd, 1H); 8.96 (dd, 1H).

### Intermediate 9: 2-Bromo-1-(5-bromolpyridin-2-yl)ethanone

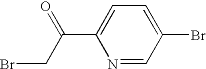

1-(5-Bromopyridin-2-yl)ethanone (Intermediate 10, WO 98/46605) (5.65 g, 28.2 mmol) was dissolved in methanol/acetic acid (50 mL+70 mL), cooled to 0° C. and 30% HBr in acetic acid (8 mL) was added. Bromine (1.45 mL, 28.3 mmol) in acetic acid (10 mL) was added dropwise and the reaction mixture was allowed to reach room temperature and then was heated to 70° C. for 1 hour. It was cooled to ˜50° C. and more bromine (0.4 mL) in acetic acid (5 mL) and methanol (15 mL) was added and it was heated to 70° C. for another 30 minutes. The reaction mixture was concentrated to dryness under reduced pressure and the residue was crystallized from isopropanol to give 3.45 g (34%) of the crude hydrobromide salt of the product as a pale yellow solid.

MS (ESP): 277.95/279.95/281.94 (MH⁺) for C₇H₅Br₂NO

¹H-NMR (L)MSO-d₆) δ: 4.96 (s, 2H); 7.93 (m, 1H); 8.29 (m, 1H); 8.87 (m, 1H); 9.19 (brs, 1H).

### Intermediate 10: 1-(5-Bromopyridin-2-yl)ethanone

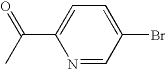 see WO98/46605

5-Bromo-2-cyanopyridine (Markevitch, David Y.; Rapta, Miroslav; Hecker, Scott J.; Renau, Thomas E.; Synth. Commun.; 33; 19; 2003; 3285-3290) (8 g, 43.7 mmol) was dissolved in dry THF (200 mL) and cooled to −20° C. Methylmagnesium bromide (43.7 mL, 3M) was added drop wise and the temperature was held between −20° C. and −10° C for 3 hours. The reaction mixture was cooled to −40° C. and conc. HCl (4.5 mL) in water (15 mL) was added dropwise. It was stirred for 10 minutes at −35° C. and then poured into a beaker with potassium phosphate buffer (300 mL, 1M, pH 7), under stirring. Ethyl acetate (300 mL) was added and the organic phase was dried over sodium sulfate. Upon concentration at room temperature under reduced pressure to ˜50 mL the product crystallized, 2.4 g, mp 112° C. The mother liquor was further concentrated and chromatographed on silica gel with dichloromethane/ethylacetate (100:1) to give another 3.25 g product (65% combined yield).

¹H-NMR (DMSO-d₆) δ: 2.60 (s, 3H); 7.88 (dd, 1H); 8.25 (dd, 1H); 8.86 (d, 1H).

### EXAMPLE 2

### (5R)-3-{3-Fluoro-4-[6-(4-morpholin-4-ylbutanoyl)pyridin-3-yl]phenyl}-5-(1H-1,2,3-triazol-1-ylmethyl)-1,3-oxazolidin-2-one

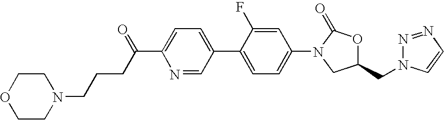

1-(5-Bromopyridin-2-yl)-4-morpholin-4-ylbutan-1-one, TFA salt (Intermediate 11) (190 mg, 0.44 mmol), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7) (173 mg, 0.44 mmol), sodium carbonate (141 mg, 1.33 mmol) and tetrakis (triphenylphospine) palladium (0) (51 mg, 0.044 mmol) were reacted as described for Example 1 to give 119 mg of the hydrochloride salt of the product (54%), as a colourless solid, mp >240° C. (dec.).

MS (ESP): 495.24 (MH⁺) for C₂₅H₂₇FN₆O₄

¹H-NMR (DMSO-d₆) δ: 2.00-2.12 (m, 2H); 3.00-4.02 (m, 13H); 4.30 (m, 1H); 4.86 (m, 2H); 5.19 (m, 1H); 7.43 (m, 1H); 7.60 (m, 1H); 7.68-7.78 (m, 2H); 8.05 (m, 1H); 8.16-8.22 (m, 2H); 8.91 (s, 1H); 10.57 (s, 1H).

The intermediate for Example 4 was prepared as follows:

### Intermediate 11: 1-(5-Bromopyridin-2-yl)-4-morpholin-4-ylbutan-1-one

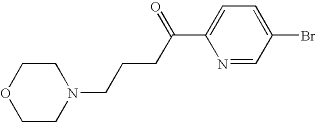

3-[(5-Bromopyridin-2-yl)carbonyl]dihydrofuran-2(3H)-one, sodium salt, (G. M. Sanders, M. van Dijk, H. C. van der Plas, Heterocycles 15, 1, 1981, 213-223) (1.05 g, 3.6 mmol) was heated in conc HCl (5 mL) for one hour at 80° C. The reaction mixture was cooled to room temperature and poured into saturated aqueous sodium hydrogencarbonate solution (100 mL). It was extracted with dichloromethane (3×100 mL), dried over sodium sulfate and solvent was removed under reduced pressure. This crude mixture of the chloride intermediate and the quinolizinium salt was heated in morpholine (3 mL) at 85° C. for 3 hours. Chromatography on silica gel with hexanes/acetone (5:1), followed by chromatography on RediSep C-18 with 5-20% acetonitrile in water (0.1% TFA) gave 190 mg of the trifluoroacetate (TFA) salt of the product (12%) as a colourless oil.

MS (ESP): 313.07/315.07 (MH⁺) for C₁₃H₁₇BrN₂O₂

¹H-NMR (DMSO-d₆) δ: 1.99 (m, 2H); 2.98-3.30 (m, 6H); 3.46 (m, 2H); 3.64 (t, 2H); 3.97 (m, 2H); 7.90 (m, 1H); 8.29 (m, 1H); 8.87 (m, 1H); 9.79 (brs, 1H).

### EXAMPLE 3

5-{2-Fluoro-4-[(5R-2-oxo-5-(1H-1,2,3-triazol-1-ylmethyl)-1,3-oxazolidin-3-yl]phenyl}pyridine-2-carbaldehyde O-(tert-butyl)oxime

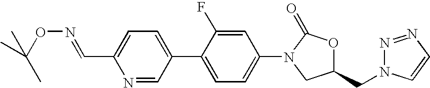

5-{2-Fluoro-4-[(5R)-2-oxo-5-(1H-1,2,3-triazol-1-ylmethyl)-1,3-oxazolidin-3-yl]phenyl}pyridine-2-carbaldehyde (Reference Example 2, 200 mg, 0.54 mmol), O-t-butyl hydroxylamine hydrochloride (89 mg, 0.71 mmol) and sodium carbonate (35 mg, 0.33 mmol) were reacted as described for Reference Example 3, but the reaction time was one hour. It was diluted with dichloromethane, washed with water and dried over sodium sulfate. Solvent was removed under reduced pressure and crystallization from ethanol/hexanes gave 260 mg of the product as a colourless solid, mp 192° C.

MS (ESP): 439.23 (MH⁺) for C₂₂H₂₃FN₆O₃

¹H-NMR (DMSO-d₆) δ: 1.34 (s, 9H); 3.95 (dd, 1H); 4.29 (dd, 1H); 4.86 (d, 2H); 5.18 (m, 1H); 7.41 (dd, 1H); 7.58 (dd, 1H); 7.67 (dd, 1H); 7.77 (s, 1H); 7.91 (d, 1H); 8.02 (m, 1H); 8.14 (s, 1H); 8.18 (s, 1H); 8.77 (s, 1H).

### REFERENCE EXAMPLE 1

### (5R)-3-[4-(6-Acetylpyridin-3-yl)-3-fluorophenyl]-5-(1H-1,2,3-triazol-1-ylmethyl)-1,3-oxazolidin-2-one

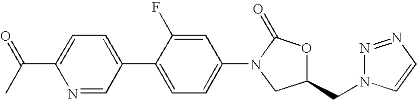

1-(5-Bromopyridin-2-yl)ethanone (Intermediate 10, 2.47 g, 12.4 mmol),), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7, 4.0 g, 10.3 mmol) and sodium carbonate (3.98 g, 37.5 mmol) were dissolved/suspended in DMF/water (50 mL, 10:1). It was degassed, flushed with nitrogen and tetrakis (triphenylphospine) palladium (0) (1.2 g, 1.03 mmol) was added. It was heated at 75° C. for 3 hours, cooled to room temperature, and the solvent was evaporated. Chromatography on silica gel with hexanes/acetone (1:1) to acetone and precipitation of the product from methanol/dichloromethane (4:1, 50 mL) by removing most of the dichloromethane under reduced pressure gave 1.98 g product (50%), as an off-white solid, mp >210° C. (dec.).

MS (ESP): 382.13 (MH⁺) for C₁₉H₁₆FN₅O₃

¹H-NMR (DMSO-d₆) δ: 2.66 (s, 3H); 3.96 (dd, 1H); 4.30 (dd, 1H); 4.86 (d, 2H); 5.19 (m, 1H); 7.43 (dd, 1H); 7.60 (dd, 1H); 7.72 (dd, 1H); 7.76 (s, 1H); 8.03 (d, 1H); 8.17 (m, 1H); 8.18 (s, 1H); 8.90 (s, 1H).

### REFERENCE EXAMPLE 2

### 5-{2-Fluoro4-[(5R)-2-oxo-5-(1H-1,2,3-triazol-1-ylmethyl)-1,3-oxazolidin-3-yl]phenyl}pyridine-2-carbaldehyde

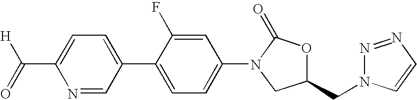

5-Bromopyridine-2-carbaldehyde (X. Wang et al, Tetrah. Left. 41 (2000), 4335) (450 mg, 2.42 mmol), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7, 939 mg, 2.42 mmol), sodium carbonate (769 mg, 7.26 mmol) and tetrakis (triphenylphospine) palladium (0) (280 mg, 0.24 mmol) were reacted as described for Example 1, but with 10 mL of solvent and 10 hours heating at 70° C. Chromatography on silica gel with hexanes/acetone (1:1) gave 535 mg (60%) of the product as a colourless solid, mp >180° C. (dec.).

MS (ESP): 368.05 (MH⁺) for C₁₈H₁₄FN₅O₃

¹H-NMR (DMSO-d₆) δ: 3.97 (dd, 1H); 4.30 (dd, 1H); 4.86 (d, 2H); 5.19 (m, 1H); 7.45 (dd, 1H); 7.61 (dd, 1H); 7.75 (dd, 1H); 7.77 (s, 1H); 8.02 (d, 1H); 8.18 (s, 1H); 8.23 (d, 1H); 9.01 (s, 1H); 10.02 (s, 1H).

### REFERENCE EXAMPLE 3

### 5-{2-Fluoro-4-[(5R)-2-oxo-5-(1H-1,2,3-triazol-1-ylmethyl)-1,3-oxazolidin-3-yl]phenyl}pyridine-2-carbaldehyde oxime

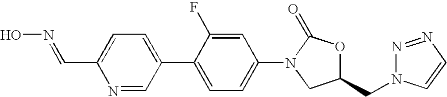

5-{2-Fluoro-4-[(5R)-2-oxo-5-(1H-1,2,3-triazol-1-ylmethyl)-1,3-oxazolidin-3-yl]phenyl}pyridine-2-carbaldehyde (Reference Example 2) (100 mg, 0.272 mmol) and hydroxylamine hydrochloride (25 mg, 0.354 mmol) were mixed in methanol/water (1:1, 5 mL), sodium carbonate (18 mg, 0.163 mmol), dissolved in water (1 mL), was added and the mixture was stirred at room temperature for one day. More hydroxylamine hydrochloride (10 mg) was added and the mixture was stirred for another 16 hours. Water (5 mL) was added, the precipitate was collected by filtration, washed with water and dried. Chromatography on silica gel with dichloromethane/methanol (15:1) and precipitation as described for Example 10 gave 41 mg (39%) of the product as a colourless solid, mp >230° C. (dec.).

MS (ESP): 382.87 (MH⁺) for C₁₈H₁₅FN₆O₃

¹H-NMR (DMSO-d₆) δ: 3.95 (dd, 1H); 4.29 (dd, 1H); 4.85 (d, 2H); 5.18 (m, 1H); 7.41 (dd, 1H); 7.58 (dd, 1H); 7.67 (dd, 1H); 7.77 (s, 1H); 7.87 (d, 1H); 8.00 (m, 1H); 8.12 (s, 1H); 8.18 (s, 1H); 8.75 (s, 1H); 11.76 (s, 1H).

### REFERENCE EXAMPLE 4

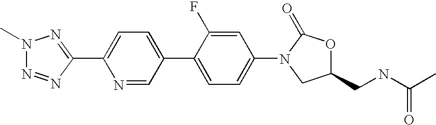

See WO 01/94342 (Dong A. Pharm. Co. Ltd) Example 139.

### REFERENCE EXAMPLE 5

### (5R)-3-(3-Fluoro-4-(6-(2-methyl-2H-tetrazol-5-yl)pyrid-3-yl)phenyl)-5-(1H-1,2,3-triazol-1-ylmethyl)-1,3-oxazolidin-2-one

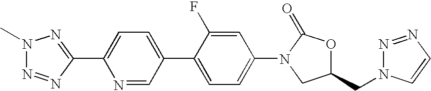

A mixture of (5R)-3-(3-fluoro-4-iodophenyl)-5-(1H-1,2,3-triazol-1-ylmethyl)-1,3-oxazolidin-2-one (Intermediate 6, 370 mg, 0.95 mmol), bis(pinacolato)diboron (605 mg, 2.4 mmol), and potassium acetate (326 mg, 3.3 mmol) in dimethylsulfoxide (5 mL) was degassed, flushed with nitrogen and trated with dichloro[1,1′]bis(diphenylphosphino)ferrocene]palladium (II) dichloromethane adduct (69 mg, 10 mol %). The mixture was heated to 80° C. for 1.5 hours, cooled to room temperature, filtered through Celite, and extracted with ethyl acetate. The organic phase was washed with aqueous ammonium chloride solution, dried over magnesium sulfate, and evaporated to dryness. The involatile residue was purified by chromatography on silica-gel [elution with hexanes:ethyl acetate (3:2)] to give a mixture of (5R)-3-(3-fluoro-4-(4,4,5,5-tetramethyl-1,3,2-dioxaborolan-2-yl)phenyl)-5-(1H-1,2,3-triazol-1-ylmethyl)-1,2-oxazolidin-2-one and the corresponding boronic acid (210 mg, ˜0.54 mmol, 57%) that was used without further purification.

A mixture of the mixture of boronate ester and boronic acid prepared above, 5-bromo-2-(2-methyl-2H-tetrazol-5-yl)pyridine (160 mg, 0.67 mmol), and potassium carbonate (448 mg, 3.24 mmol) in N,N-dimethyl formamide and water (10 mL, 7:1) was degassed, flushed with nitrogen, and treated with tetrakis (triphenylphospine) palladium (0) (62 mg, 0.054 mmol). The reaction mixture was heated at 80° C. for 1.5 hours, cooled to room temperature, filtered through Celite, extracted with ethyl acetate, dried over magnesium sulfate, and evaporated to dryness. The involatile residue was purified by chromatography on silica-gel [elution with ethyl acetate:hexanes (3:2)] to give the product as a colorless amorphous solid (140 mg, 61%).

MS (ESP): 422.47 (MH⁺) for C₁₉H₁₆FN₉O₂

¹H-NMR (DMSO-d₆) δ: 3.98 (dd, 1H); 4.31 (dd, 1H); 4.47 (s, 3H); 4.86 (m, 2H); 5.18 (m, 1H); 7.45 (m, 1H); 7.61 (m, 1H); 7.74 (m, 1H); 7.77 (brs, 1H); 8.12-8.27 (m, 3H); 8.93 (s, 1H).

The intermediates for this Reference Example were prepared as follows:

### 5-Bromo-2-tetrazol-5-ylpyridine

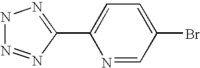

A mixture of 3-bromo-6-cyano-pyridine (2 g, 10.9 mmol), sodium azide (0.85 g, 13 mmol), and ammonium chloride (0.59 g, 11 mmol) in N,N-dimethylformamide (20 mL) was heated for 1 h at 120° C. The reaction mixture was diluted with ethyl acetate (˜100 mL) and the product was isolated by filtration and then washed with ethyl acetate to give the title compound, an off-white amorphous solid which was used in the next step without further purification.

### 5-Bromo-2-(2-methyl-2H-tetrazol-5-yl)pyridine and 5-bromo-2-(1-methyl-1H-tetrazol-5-yl)pyridine

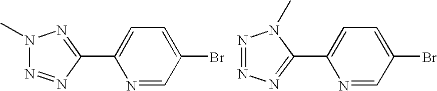

5-Bromo-2-(2-methyl-2H-tetrazol-5-yl)pyridine and 5-bromo-2-(1-methyl-1H-tetrazol-5-yl)pyridine were prepared according to the procedure described by Dong A Pharmaceuticals (WO 01/94342).

A mixture of 6.5 g unpurified 5-bromo-2-tetrazol-5-ylpyridine [Dong A Pharmaceuticals (WO 01/94342)] (˜28 mmol) and sodium hydroxide (9 g, 125 mmol) in dry DMF was evaporated to dryness under reduced pressure. A stirred solution of the involatile residue in dry DMF (50 mL) was treated dropwise at ice-bath temperature with iodomethane (3.0 mL, 48 mmol). The stirred reaction mixture was allowed to warm and then maintained at room temperature for 2 hours. The reaction mixture was partitioned between iced water and ethyl acetate. The organic phase was washed with water, dried over magnesium sulfate, and then evaporated under reduced pressure to give a residue that was purified by chromatography on silica gel [elution with dichloromethane:ethyl acetate (60:1)] to give:
1. 5-bromo-2-(1-methyl-1H-tetrazol-5-yl)pyridine (1.397 g), a colorless solid, (TLC: silica-gel, hexanes:ethyl acetate (4:1), Rf: 0.3), ¹H-NMR (DMSO-d₆) (300 MHz) δ: 4.38 (s, 3H); 8.17 (d, 1H); 8.35 (dd, 1H); 8.96 (d, 1H).
2. 5-bromo-2-(2-methyl-2H-tetrazol-5-yl)pyridine (1.07 g), a colorless solid, (TLC: silica-gel, hexanes:ethyl acetate (4:1), Rf: 0.1). ¹H-NMR (DMSO-d₆) (300 MHz) δ: 4.46 (s, 3H); 8.09 (d, 1H); 8.28 (dd, 1H); 8.88 (d, 1H).

Structure assignment based on nmr HMBC (Heteronuclear Multiple Bond Correlation) experiments, in which long range coupling of the protons of CH3 to the C5 of the tetrazole ring is observed in the 1-methyl-1H-isomer of Rf 0.3, but not in the 2-methyl-2H-isomer of Rf 0.1). The compound referred to as 5-bromo-2-(1-methyl-1H-tetrazol-5-yl)pyridine is thus the isomer of Rf 0.3 and the compound referred to as 5-bromo-2-(2-methyl-2H-tetrazol-5-yl)pyridine is thus the isomer of Rf 0.1

### EXAMPLE 4

### (5R-3-(3-Fluoro-4-{6-[(2-methyl-1H-imidazol-1-yl)acetyl]pyridin-3-yl}phenyl)-5-(1H-1,2,3-triazol-1-ylmethyl)-1,3-oxazolidin-2-one

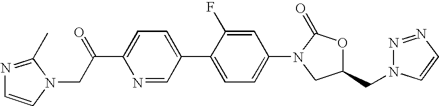

1-(5-Bromopyridin-2-yl)-2-(2-methyl-1H-imidazol-1-yl)ethanone (Intermediate 12) (336 mg, 1.2 mmol), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7, 388 mg, 1.0 mmol), sodium carbonate (300 mg, 2.8 mmol) and tetrakis (triphenylphospine) palladium (0) (115 mg, 0.1 mmol) were reacted like described under Example 1 to give 276 mg (55%) of product as the hydrochloride salt, colourless solid, mp >240° C. (dec.).

MS (ESP): 462.29 (MH⁺) for C₂₃H₂₀FN₇O₃

¹H-NMR (DMSO-d₆) δ: 2.55 (s, 3H); 3.97 (m, 1H); 4.31 (m, 1H); 4.87 (d, 2H); 5.20 (m, 1H); 6.05 (s, 2H); 7.46 (dd, 1H); 7.57-7.65 (m, 3H); 7.72-7.80 (m, 2H); 8.13 (d, 1H); 8.19 (s, 1H); 8.27 (m, 1H); 9.03 (s, 1H); 14.48 (brs, 1H).

The intermediate for Example 4 were prepared as follows:

### Intermediate 12: 1-(5-Bromopyridin-2-yl)-2-(2-methyl-1H-imidazol-1-yl)ethanone

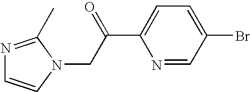

2-Bromo-1-(5-bromopyridin-2-yl)ethanone hydrobromide (Intermediate 9, 650 mg, 1.8 mmol) was suspended in dry THF (6 mL) and cooled to 0° C. 2-Methylimidazole (593 mg, 7.2 mmol) was added and the mixture was stirred for 2 hour. The reaction mixture was diluted with dichloromethane, washed with water, and dried over sodium sulfate. Chromatography on silica gel with dichloromethane/methanol (20:1) gave 336 mg of the product (66%), as an off-white solid.

MS (ESP): 280.07/282.07 (MH⁺) for C₁₁H₁₀BrN₃O

¹H-NMR (DMSO-d₆)δ: 2.15 (s, 3H); 5.68 (s, 2H); 6.74 (d, 1H); 7.00 (d, 1H); 7.93 (d, 1H); 8.33 (dd, 1H); 8.96 (d, 1H).

### EXAMPLE 5

### (5R)-3-(3-Fluoro-4-{6-[(4-methyl-1H-imidazol-1-yl)acetyl]pyridin-3-yl}phenyl)-5-(1H-1,2,3-triazol-1-ylmethyl)-1,3oxazolidin-2-one

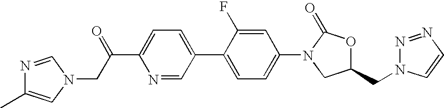

1-(5-Bromopyridin-2-yl)-2-(4-methyl-1H-imidazol-1-yl)ethanone (Intermediate 13) (170 mg, 0.607 mmol), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7, 214 mg, 0.552 mmol), sodium carbonate (175 mg, 1.65 mmol) and tetrakis (triphenylphospine) palladium (0) (64 mg, 0.05 mmol) were reacted like described under Example 1 to give 155 mg (56%) of product as the hydrochloride salt, colourless solid, mp >217° C. (dec.).

MS (ESP): 462.29 (MH⁺) for C₂₃H₂₀FN₇O₃

¹H-NMR (DMSO-d₆) δ: 2.32 (s, 3H); 3.97 (dd, 1H); 4.31 (dd, 1H); 4.87 (d, 2H); 5.20 (m, 1H); 6.06 (s, 2H); 7.40-7.49 (m, 2H); 7.62 (dd, 1H); 7.74-7.80 (m, 2H); 8.13 (d, 1H); 8.19 (s, 1H); 8.28 (m, 1H); 8.95 (s, 1H); 9.02 (brs, 1H); 14.57 (brs, 1H).

The intermediate for Example x were prepared as follows:

### Intermediate 13: 1-(5-Bromopyridin-2-yl)-2-(4-methyl-1H-imidazol-1-yl)ethanone

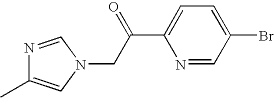

2-Bromo-1-(5-bromopyridin-2-yl)ethanone hydrobromide (Intermediate 9, 1.2 g, 3.3 mmol) was suspended in dry THF (20 mL) and cooled to 0° C. 4-Methylimidazole (1.09 g, 13.3 mmol) was added and the mixture was stirred for 2 hour. The reaction mixture was diluted with dichloromethane, washed with water, and dried over sodium sulfate. Chromatography on silica gel with acetone/hexanes (1:1 to 2:1) gave 170 mg of the product (18%) as an off-white solid (rf=0.29, TLC: acetone/hexanes 1:1; the 5-methylimidazole analog was also formed, rf=0.21).

MS (ESP): 280.07/282.07 (MH⁺) for C₁₁H₁₀BrN₃O

¹H-NMR (DMSO-d₆) δ: 2.09 (s, 3H); 5.67 (s, 2H); 6.79 (s, 1H); 7.44 (s, 1H); 7.93 (d, 1H); 8.33 (dd, 1H); 8.94 (d, 1H). The assignment of the 4- and 5-methylimidazole isomers was based on HMBC (heteronuclear multiple bond correlation NMR-experiment).

### EXAMPLE 6

### (5R)-3-{3-Fluoro-4-[6(piperidin-4-ylacetyl)pyridin-3-yl]phenyl}-5-(1H-1,2,3-triazol-1-ylmethyl)-1,3-oxazolidin-2-one

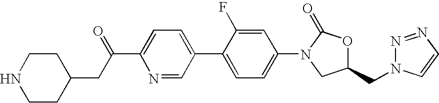

A solution of tert-butyl 4-[2-(5-{2-fluoro-4-[(5R)-2-oxo-5-(1H-1,2,3-triazol-1-ylmethyl)-1,3-oxazolidin-3-yl]phenyl}pyridin-2-yl)-2-oxoethyl]piperidine-1-carboxylate (Intermediate 14, 630 mg, 1.12 mmol) in dioxane (5 mL) was treated under vigorous stirring with a solution of HCl in dioxane (4M, 5 mL). The reaction was stirred over night at room temperature, solvent was evaporated under reduced pressure and the residue recrystallized from water/isopropanol (33 mL, 1:10) to give 409 mg (73%) of product as the hydrochloride salt, colourless solid, mp >196° C. (dec.).

MS (ESP): 465 (MH⁺) for C₂₄H₂₅FN₆O₃

¹H-NMR (DMSO-d₆) δ: 1.45 (m, 2H); 1.83 (m, 2H); 2.19 (m, 1H); 2.88 (m, 2H); 3.16-3.26 (m, 4H); 3.97 (dd, 1H); 4.30 (dd, 1H); 4.87 (d, 2H); 5.20 (m, 1H); 7.43 (dd, 1H); 7.60 (dd, 1H); 7.72 (dd, 1H); 7.77 (s, 1H); 8.05 (d, 1H); 8.16-8.21 (m, 1H); 8.19 (s, 1H); 8.70 (m, 1H); 8.87-8.98 (m, 1H); 8.90 (brs, 1H).

The intermediates for Example 6 were prepared as follows:

### Intermediate 14: tert-Butyl 4-[2-(5-{2-fluoro-4-[(5R)-2-oxo-5-(1H-1,2,3-triazol-1ylmethyl)-1,3-oxazolidin-3-yl]phenyl}pyridin-2-yl)-2-oxoethyl]piperidine-1-carboxylate

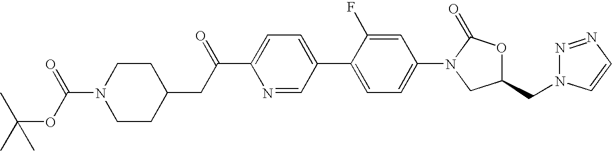

tert-Butyl 4-[2-(5-bromopyridin-2-yl)-2-oxoethyl]piperidine-1-carboxylate (Intermediate 15) (590 mg, 1.54 mmol), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7, 598 mg, 1.54 mmol), sodium carbonate (490 mg, 4.6 mmol) and tetrakis (triphenylphospine) palladium (0) (178 mg, 0.154 mmol) were reacted like described under Example 1. Chromatography on silica gel with hexanes/acetone (2:1) gave 691 mg (79%) of product as a colourless solid, mp >140° C.

MS (ESP): 565.66 (MH⁺) for C₂₉H₃₃FN₆O₅

¹H-NMR (DMSO-d₆) δ: 1.11 (m, 2H); 1.38 (s, 9H); 1.66 (m, 2H); 2.07 (m, 1H); 2.72 (m, 2H); 3.13 (m, 2H); 3.84-4.00 (m, 3H); 4.30 (dd, 1H); 4.86 (d, 2H); 5.19 (m, 1H); 7.43 (dd, 1H); 7.60 (dd, 1H); 7.72 (dd, 1H); 7.77 (s, 1H); 8.05 (d, 1H); 8.15-8.21 (m, 1H); 8.18 (s, 1H); 8.90 (brs, 1H).

### Intermediate 15: tert-Butyl 4-[2-(5-bromopyridin-2-yl)-2-oxoethyl]piperidine-1-carboxylate

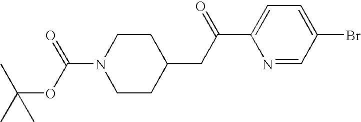

A solution of 5-bromo-2-iodopyridine (764 mg, 2.69 mmol) in THF (10 mL) was cooled to −10° C. and treated dropwise with a solution of iPrMgCl in THF (2M, 1.35 mL, 2.63 mmol). The reaction mixture was stirred for 30 minutes and then a solution of tert-butyl 4-{2-[methoxy(methyl)amino]-2-oxoethyl}piperidine-1-carboxylate (Intermediate 16, 770 mg, 2.69 mmol) in THF (5 mL) was added via syringe. The reaction mixture was allowed to warm to 10° C. over 2 hours and was then poured into potassium phosphate buffer (1M, pH 7, 200 mL) under stirring. It was extracted with ethyl acetate (150 mL) and dried over sodium sulfate. Chromatography on silica gel with hexanes/ethyl acetate (7:1) gave 591 mg (57%) of the product as a colourless solid.

MS (ESP): 383.47/385.47 (MH⁺) for C₁₇H₂₃BrN₂O₃

¹H-NMR (DMSO-d₆) δ: 1.09 (m, 2H); 1.37 (s, 9H); 1.63 (m, 2H); 2.02 (m, 1H); 2.72 (m, 2H); 3.06 (m, 2H); 3.91 (m, 2H); 7.87 (d, 1H); 8.25 (dd, 1H); 8.85 (d, 1H).

### Intermediate 16: Tert-butyl 4-{2-methoxy(methyl)amino]-2-oxoethyl}piperidine-1-carboxylate

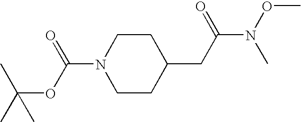

To a solution of boc-(4-carboxymethyl)-piperidine (M. S. Egbertson et al, J. Med. Chem. 37 (16), 2537-2551(1994)) (1 g, 4.11 mmol) and bis (2-oxo-3-oxazolidinyl)phosphonic chloride (1.05 g, 4.11 mmol) in DMF (4 mL) was added N,O-dimethylhydroxylamine hydrochloride (561 mg, 5.57 mmol), followed by diisopropylethyl amine (2.15 mL, 12.3 mmol). The reaction mixture was stirred for 30 minutes, diluted with ethyl acetate (100 mL), washed with water (2×50 mL) and dried over sodium sulfate. Chromatography on silica gel with hexanes/ethyl acetate (2:1 to 1:2) gave 770 mg (65%) of the product as a colourless oil.

MS (ESP): 187.25 (M-bocH⁺) for C₁₄H₂₆N₂O₄

¹H-NMR (DMSO-d₆) δ: 1.02 (m, 2); 1.37 (s, 9H); 1.61 (m, 2H); 1.85 (m, 1H); 2.29 (m, 2H); 2.68 (m, 2H); 3.06 (s, 3H); 3.62 (s, 3H); 3.89 (m, 2H).

### EXAMPLE 7

### (5R)-3-(3-Fluoro-4-{6-[4-(4methylpiperazin-1-yl)butanoyl]pyridin-3-yl}phenyl)-5-(1H-1,2,3-triazol-1-ylmethyl)-1,3-oxazolidin-2-one

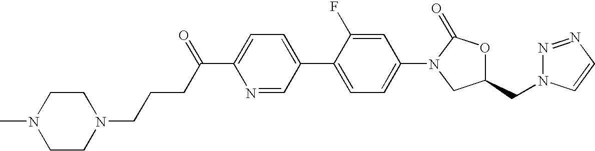

1-(5-Bromopyridin-2-yl)-4-(4-methylpiperazin-1-yl)butan-1-one (Intermediate 17, 580 mg, 1.8 mmol), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7,690 mg, 1.8 mmol), sodium carbonate (377 mg, 3.55 mmol) and tetrakis (triphenylphospine) palladium (0) (205 mg, 0.18 mmol) were reacted like described under Example 1, but using 10 mL solvent. Chromatography on silica gel with dichloromethane/methanol (5:1 to 3:1) gave 510 mg (57%) of product as a colourless hard foam. The free base of the product (310 mg, 0.61 mmol) was taken up in isopropanol (10 mL), HCl (1M in ether, 2 mL) was added under vigorous stirring. After stirring for 15 minutes, solvent was removed under reduced pressure and the residue was crystallized from water/isopropanol (˜30 mL, 1:15) to give 261 mg of the bis hydrochloride salt of the product as a colourless solid, mp >240° C.

MS (ESP): 508 (MH⁺) for C₂₆H₃₀FN₇O₃

¹H-NMR (DMSO-d₆) δ: 2.09 (m, 2H); 2.82 (s, 3H); 3.13-3.88 (m, 12H); 3.97 (dd, 1H); 4.30 (dd, 1H); 4.86 (d, 2H); 5.19 (m, 1H); 7.43 (m, 1H); 7.60 (m, 1H); 7.73 (dd, 1H); 7.77 (s, 1H); 8.06 (d, 1H); 8.15-8.23 (m, 2H); 8.91 (brs, 1H).

The intermediates for Example 7 were prepared as follows:

### Intermediate 17: 1-(5-Bromopyridin-2-yl)-4-(4-methylpiperazin-1-yl)butan-1-one

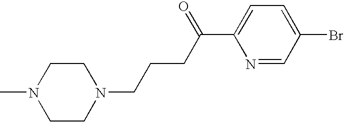

5-Bromo-2-iodopyridine (927 mg, 3.27 mmol) was reacted with isopropyl magnesium chloride (2M in THF, 1.64 mL, 3.27 mmol) and N-methoxy-N-methyl-4-(4-methylpiperazin-1-yl)butanamide (Intermediate 18, 749 mg, 3.27 mmol) like described for Intermediate 15. Chromatography on silica gel with dichloromethane/methanol (10:1 to 4:1) gave 582 mg (55%) of the product as a colourless oil.

MS (ESP): 326.47/328.47 (MH⁺) for C₁₄H₂₀BrN₃O

¹H-NMR (DMSO-d₆) δ: 1.80 (tt, 2H); 2.04 (s, 3H); 2.02-2.35 (m, 10H); 3.07 (t, 2H); 7.87 (d, 1H); 8.25 (m, 1H); 8.85 (m, 1H).

### Intermediate 18: N-methoxy-N-methyl-4-(4-methylpiperazin-1-yl)butanamide

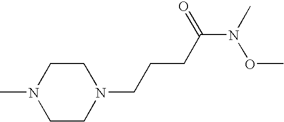

4-Chloro-N-methoxy-N-methyl-butyramide (V. Selvamurugan et al, Synthesis 15, 2239-2246, 2001) (1 g, 6.04 mmol) and 1-methylpiperazine (1.2 g, 12 mmol) were mixed in DMSO (2 mL) and heated to 80° C. for 3 hours. The reaction was quenched with saturated aqueous sodium hydrogencarbonate solution (˜100 mL), extracted with dichloromethane (3×100 mL) and dried over sodium sulfate. Chromatography on silica gel with dichloromethane/methanol (18:1 to 3:1) gave 749 mg (54%) of the product as a slightly yellow oil.

MS (ESP): 326.47/328.47 (MH⁺) for C₁₁H₂₃N₃O₂

¹H-NMR (DMSO-d₆) δ: 1.63 (tt, 2H); 2.13 (s, 3H); 2.10-2.45 (m, 10H); 3.07 (s, 3H); 3.39 (m, 2H); 3.64 (s, 3H).

### EXAMPLE 8

### (5R)-3-{3-Fluoro-4-[6-(piperidin-4-ylcarbonyl)pyridin-3-yl]phenyl}-5-(1H-1,2,3-triazol-1-ylmethyl)-1,3-oxazolidin-2-one

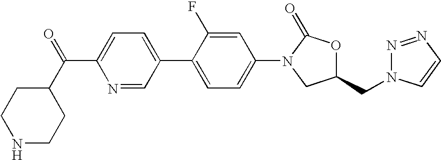

Benzyl 4-[(5-{2-fluoro-4-[(5R)-2-oxo-5-(1H-1,2,3-triazol-1-ylmethyl)-1,3-oxazolidin-3-yl]phenyl}pyridin-2-yl)carbonyl]piperidine-1-carboxylate (Intermediate 19, 305 mg, 0.52 mmol) was hydrogenated on Pd/C (10%, wet) in methanol (10 mL, containing a few drops acetic acid) at room temperature and normal pressure for 2 hours. The resulting mixture was filtered through a 0.45 μM membrane and solvent was removed under reduced pressure. Chromatography was performed on C-18 RP silica (using a RediSep cartridge) with 0-20% acetonitrile (containing 0.1% TFA) in water. Fractions containing product were pooled and solvent was removed under reduced pressure. The residue was taken up in isopropanol (20 mL) and HCl (1M in ether, 0.5 mL) was added under vigorous stirring. It was diluted with hexanes (10 mL) and the solid was collected by filtration and dried at 50° C. under reduced pressure to give 71 mg (28%) of the hydrochloride salt of the product as a colourless solid, mp >275° C. (dec.).

MS (ESP: 451.27 (MH⁺) for C₂₃H₂₃FN₆O₃

¹H-NMR (DMSO-d₆) δ: 1.79 (m, 2H); 2.04 (m, 2H); 3.10 (m, 2H); 3.97 (dd, 1H); 4.12 (m, 1H); 4.30 (dd, 1H); 4.86 (d, 2H); 5.19 (m, 1H); 7.44 (dd, 1H); 7.60 (dd, 1H); 7.73 (dd, 1H); 7.77 (s, 1H); 8.07 (d, 1H); 8.18 (s, 1H); 8.21 (m, 1H); 8.50 (m, 1H); 8.76 (m, 1H); 8.92 (brs, 1H); an additional 2H are either under the solvent or under the HDO signal.

The intermediates for Example 8 were prepared as follows: Intermediate 19: Benzyl 4-[(5-{2-fluoro-4-[(5R)-2-oxo-5-(1H-1,2,3-triazol-1-ylmethyl)-1,3-oxazolidin-3-yl]phenyl}pyridin-2-yl)carbonyl]piperidine-1-carboxylate

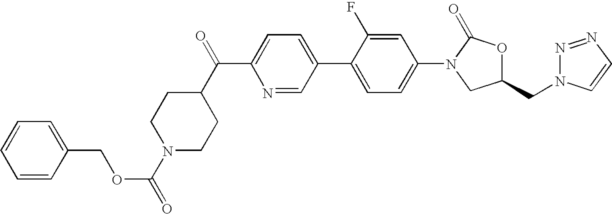

Benzyl 4-[(5-bromopyridin-2-yl)carbonyl]piperidine-1-carboxylate (Intermediate 20, 250 mg, 0.62 mmol), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7, 241 mg, 0.62 mmol), sodium carbonate (197 mg, 1.86 mmol) and tetrakis (triphenylphospine) palladium (0) (72 mg, 0.06 mmol) were reacted like described under Example 1. Chromatography on silica gel with hexanes/acetone (1:1) gave 339 mg (93 %) of product as a slightly yellow hard foam.

MS (ESPE: 585.41 (MH⁺) for C₃₁H₂₉FN₆O₅

¹H-NMR (DMSO-d₆) δ: 1.47 (m, 2H); 1.87 (m, 2H); 3.01 (m, 2H); 3.93-4.12 (m, 4H); 4.30 (dd, 1H); 4.86 (d, 2H); 5.08 (s, 2H); 5.19 (m, 1H); 7.28-7.46 (m, 6H); 7.60 (dd, 1H); 7.73 (dd, 1H); 7.77 (s, 1H); 8.06 (d, 1H); 8.15-8.23 (m, 2H); 8.93 (brs, 1H).

### Intermediate 20: Benzyl 4-[(5-bromopyridin-2-yl)carbonyl]piperidine-1-carboxylate

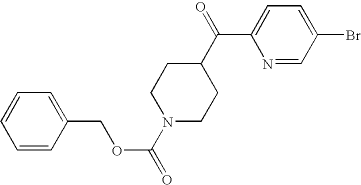

5-Bromo-2-iodopyridine (490 mg, 1.73 mmol) was reacted with isopropyl magnesium chloride (2M in THF, 0.86 mL, 1.73 mmol) and benzyl 4-{[methoxy(methyl)amino]carbonyl}piperidine-1-carboxylate (Intermediate 21, 529 mg, 1.73 mmol) like described for Intermediate 15. Chromatography on silica gel with hexanes/ethylacetate (5:1) gave 252 mg (36%) of the product as a colourless solid.

MS (ESP): 403.19/405.19 (MH⁺) for C₁₉H₁₉BrN₂O₃

¹H-NMR (DMSO-d₆) δ: 1.42 (m, 2H); 1.83 (m, 2H); 2.99 (m, 2H); 3.93 (m, 1H); 4.04 (m, 2H); 5.07 (s, 2H); 7.26-7.40 (m, 5H); 7.90 (d, 1H); 8.28 (d, 1H); 8.88 (d, 1H).

### Intermediate21: Benzyl 4-{methoxy(methyl)amino]carbonyl}piperidine-1-carboxylate

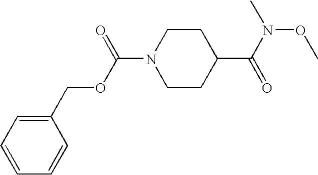

A mixture of benzyl 4-(chlorocarbonyl)piperidine-1-carboxylate (H. Harada et al, Bioorg. Med. Chem. Lett. 12 (6), 967-970, 2002) (1 g, 3.55 mmol) and N,O-dimethylhydroxylamine hydrochloride (346 mg, 3.55 mmol) in dichloromethane (20 mL) was cooled to −20° C. and pyridine (1.15 mL, 14.2 mmol) was added. The reaction mixture was allowed to reach room temperature over 30 minutes and was stirred for another hour. It was diluted with dichloromethane (100 mL), washed with potassium phosphate buffer (1M, pH 7, 3×100 mL) and dried over sodium sulfate. Chromatography on silica gel with hexanes/ethyl acetate (1:1 to 0:1) gave 650 mg (60%) of the product as a colourless oil.

MS (ESP): 307.25 (MH⁺) for C₁₆H₂₂N₂O₄

¹H-NMR (DMSO-d₆) δ: 1.40 (m, 2H); 1.66 (m, 2H); 2.80-2.99 (m, 3H); 3.08 (s, 3H); 3.67 (s, 3H); 4.01 (m, 2H); 5.06 (s, 2H); 7.26-7.45 (m, 5H).

### EXAMPLE 9

### (5R)-3-{4-[6-(Azetidin-3-ylcarbonyl)pyridin-3-yl]-3-fluorophenyl}-5-(1H-1,2,3-triazol-1-ylmethyl)-1,3-oxazolidin-2-one

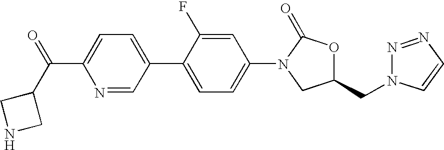

A solution of tert-butyl 3-[(5-{2-fluoro-4-[(5R)-2-oxo-5-(1H-1,2,3-triazol-1-ylmethyl)-1,3-oxazolidin-3-yl]phenyl}pyridin-2-yl)carbonyl]azetidine-1-carboxylate (Intermediate 22, 220 mg, 0.42 mmol) in dioxane (2 mL) was treated under vigorous stirring with HCl (4M in dioxane, 2 mL) and it was stirred at room temperature for one hour. Solvent was evaporated under reduced pressure. Chromatography was performed on C-18 RP silica (using a RediSep cartridge) with 0-20% acetonitrile (containing 0.1% TFA) in water. Fractions containing product were pooled and solvent was removed under reduced pressure. The residue was taken up in isopropanol (20 mL) and HCl (1M in ether, 0.2 mL) was added under vigorous stirring. The precipitated solid was collected by filtration and dried at room temperature under reduced pressure to give 44 mg (23%) of the hydrochloride salt of the product as a greenish solid, mp >90° C. (dec).

MS (ESP): 423.25 (MH⁺) for C₂₁H₁₉FN₆O₃

¹H-NMR (DMSO-d₆) δ: 3.97 (dd, 1H); 4.17-4.34 (m, 5H); 4.70 (m, 1H); 4.86 (d, 2H); 5.19 (m, 1H); 7.44 (m, 1H); 7.60 (m, 1H); 7.73 (dd, 1H); 7.77 (s, 1H); 8.11-8.27 (m, 3H); 8.81 (m, 1H); 8.90 (brs, 1H); 9.22 (m, 1H).

The intermediates for Example 9 were prepared as follows:

### Intermediate 22: tert-Butyl 3-[(5-{2-fluoro-4-[(5R)-2-oxo-5-(1H-1,2,3-triazol-1-ylmethyl)-1,3-oxazolidin-3-yl]phenyl}pyridin-2-ylcarbonyl]azetidine-1-carboxylate

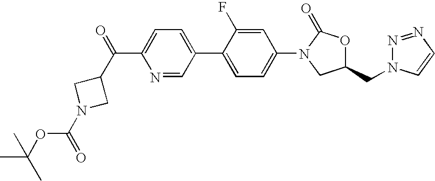

tert-Butyl 3-[(5-bromopyridin-2-yl)carbonyl]azetidine-l-carboxylate (Intermediate 23, 447 mg, 1.31 mmol), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7, 509 mg, 1.31 mmol), sodium carbonate (416 mg, 3.93 mmol) and tetrakis (triphenylphospine) palladium (0) (151 mg, 0.13 mmol) were reacted like described under Example 1, but using 10 mL of solvent. Chromatography on silica gel with hexanes/acetone (1:1) gave 500 mg (73%) of product as a colourless hard foam.

MS (ESP): 523.37 (MH⁺) for C₂₆H₂₇FN₆O₅

¹H-NMR (DMSO-d₆) δ: 1.37 (s, 9H); 3.93-4.18 (m, 5H); 4.30 (dd, 1H); 4.53 (m, 1H); 4.86 (d, 2H); 5.19 (m, 1H); 7.44 (dd, 1H); 7.60 (dd, 1H); 7.73 (dd, 1H); 7.77 (s, 1H); 8.10 (d, 1H); 8.18 (s, 1H); 8.20 (m, 1H); 8.88 (brs, 1H).

### Intermediate 23: tert-Butyl 3-[(5-bromopyridin-2-yl)carbonyl]azetidine-1-carboxylate

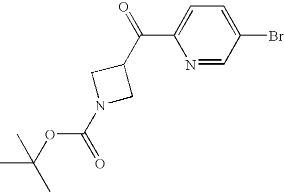

5-Bromo-2-iodopyridine (1.09 g, 3.85 mmol) was reacted with isopropyl magnesium chloride (2M in THF, 1.83 mL, 3.66 mmol) and tert-butyl 3-{[methoxy(methyl)amino]carbonyl}azetidine-1-carboxylate (Intermediate 24, 466 mg, 1.9 mmol) like described for Intermediate 15. Chromatography on silica gel with hexanes/ethylacetate (7:1) gave 447 mg (69%) of the product as a colourless solid.

MS (ESP): 341.11/343.11 (for C₁₄H₁₇BrN₂O₃

¹H-NMR (DMSO-d₆) δ: 1.36 (s, 9H); 3.92-4.14 (m, 4H); 4.44 (m, 1H); 7.93 (d, 1H); 8.28 (dd, 1H); 8.84 (d, 1H).

### Intermediate 24: tert-Butyl 3-{[methoxy(methyl)amino]carbonyl}azetidine-1-carboxylate

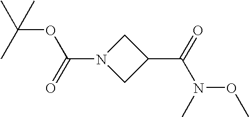

To a solution of 1-(tert-butoxycarbonyl)azetidine-3-carboxylic acid (H. Itani et al, Biorg. Med. Chem. Lett. 12 (5), 757-762, 2002) (0.5 g, 2.48 mmol) and bis (2-oxo-3-oxazolidinyl)phosphonic chloride (0.633 g, 2.48 mmol) in DMF (4 mL) was added N,O-dimethylhydroxylamine hydrochloride (339 mg, 3.48 mmol), followed by diisopropylethyl amine (1.3 mL, 7.45 mmol). The exothermic reaction was allowed to cool to room temperature and was stirred for 2 hours. Excess base was removed under reduced pressure, the residue was diluted with ethyl acetate (100 mL), washed with potassium phosphate buffer (1M, pH 7, 2×100 mL) and with water (2×100 mL) and dried over sodium sulfate. Chromatography on silica gel with hexanes/acetone (3:1) gave 470 mg (77%) of the product as a colourless solid.

MS (ESP): 267.23(MNa⁺) for C₁₁H₂₀N₂O₄

¹H-NMR (DMSO-d₆) δ: 1.36 (s, 9H); 3.10 (s, 3H); 3.61 (s, 3H); 3.68 (m, 1H); 3.83-4.00 (m, 4H).

### EXAMPLE 10

### (5R)-3-(4-{6-[(2,5-Dimethyl-1H-imidazol-1-yl)acetyl]pyridin-3-yl}-3-fluorophenyl)-5-(1H-1,2,3-triazol-1-ylmethyl)1,3-oxazolidin-2-one

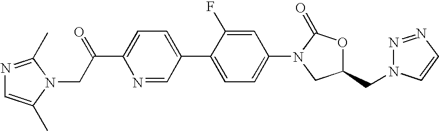

1-(5-Bromopyridin-2-yl)-2-(2,5-dimethyl-1H-imidazol-1-yl)ethanone (Intermediate 25, 985 mg, 3.35 mmol), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7, 1.30 g, 3.35 mmol), sodium carbonate (887 mg, 8.37 mmol) and tetrakis (triphenylphospine) palladium (0) (387 mg, 0.35 mmol) were reacted like described under Example 1, but using 15 mL of solvent and heating at 75° C. for 6 hours. Chromatography on silica gel with dichloromethane/methanol (10:1 to 8:1), followed by crystallization from ethanol, gave 658 mg (41%) of product as colourless needles, mp 112-115° C.

MS (ESP): 476.16 (MH⁺) for C₂₄H₂₂FN₇O₃

¹H-NMR (DMSO-d₆) δ: 2.01 (s, 3H); 2.15 (s, 3H); 3.97 (dd, 1H); 4.31 (dd, 1H); 4.86 (d, 2H); 5.19 (m, 1H); 5.64 (s, 2H); 6.51 (s, 1H); 7.46 (dd, 1H); 7.62 (dd, 1H); 7.75 (dd, 1H); 7.77 (s, 1H); 8.10 (d, 1H); 8.19 (s, 1H); 8.25 (m, 1H); 8.99 (brs, 1H).

The intermediates for Example 10 were prepared as follows:

### Intermediate 25: 1-(5-Bromopyridin-2-yl)-2-(2,5-dimethyl-1H-imidazol-1-yl)ethanone

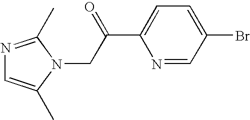

1-[2-(5-Bromopyridin-2-yl)-2-oxoethyl]-2,5-dimethyl-3-trityl-1H-imidazol-3-ium bromide (Intermediate 26, 4.75 g, 7.7 mmol) was dissolved in dichloromethane (100 mL) and trifluoroacetic acid (15 mL) was added. The mixture was heated at gentle reflux for 1.5 hours. It was diluted with dichloromethane (200 mL), washed with potassium phosphate buffer (pH 7, 1M, ˜600 mL), the aqueous phase was extracted three times with dichloromethane (3×100 mL) and the combined organic phases were dried over sodium sulfate. Chromatography on silica gel with dichloromethane/methanol (20:1) gave 1.938 g (77%) of the product as an off-white solid.

MS (ESP): 294/296 (MH⁺) for C₁₂H₁₂BrN₃O

¹H-NMR (DMSO-d₆) δ: 1.98 (s, 3H); 2.12 (s, 3H); 5.57 (s, 2H); 6.49 (s, 1H); 7.93 (m, 1H); 8.35 (m, 1H); 8.98 (m, 1H).

### Intermediate 26: 1-[2-(5-Bromopyridin-2-yl)-2-oxoethyl]-2,5-dimethyl-3-trityl-1H-imidazol-3-ium bromide

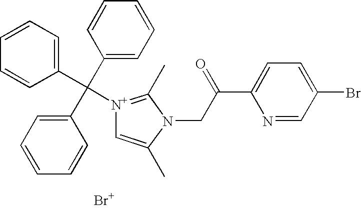

A mixture of 2,4-dimethyl-1-trityl-1H-imidazole (Intermediate 27,4.5 g, 13.4 mmol2-bromo-1-(5-bromopyridin-2-yl)ethanone (free base of Intermediate 9, 2.5 g, 9 mmol) (the free base was generated from the hydrobromide salt by treating a suspension of Intermediate 9 in ethyl acetate with potassium phosphate buffer (pH 7, 1M), washing the organic phase with water and drying over sodium sulfate) and 2,6-di-tert-butylpyridine (3 mL, 13.35 mmol) were heated in 1,4-dioxane (50 mL) at 75° C. for 30 minutes. The reaction mixture was allowed to cool to room temperature, the precipitate was collected by filtration and washed with hexanes (2×50 mL) to give 4.75 g (86 %) of the product as an off-white solid, mp >150° C. (dec).

MS (ESP): 535.95/537.95 (MH⁺) for C₃₁H₂₇BrN₃O

¹H-NMR (DMSO-d₆) δ: 1.82 (s, 3H); 2.21 (s, 3H); 5.95 (s, 2H); 7.07 (s, 1H); 7.12-7.18 (m, 6H); 7.44-7.65 (m, 9H); 7.98 (m, 1H); 8.36 (m, 1H); 8.98 (m, 1H).

### Intermediate 27: 2,4-Dimethyl-1-trityl-1H-imidazole

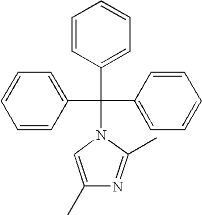

A solution of trityl chloride (15 g, 55 mmol) in dichloromethane (50 mL) was added drop wise over 45 minutes to a solution of 2,4-dimethyl imidazole (5 g, 52 mmol) in a mixture of dichloromethane (100 mL) and triethylamine (11.3 mL, 81 mmol) at room temperature. The mixture was stirred over night, then quenched with methanol (4 mL) and stirred for additional 30 minutes. The solvent was evaporated, the residue taken up in toluene (600 mL), washed with potassium phosphate buffer (pH 7, 1M, 2×200 mL) and with water (200 mL). The organic phase was diluted with dichloromethane (200 mL), dried over sodium sulfate, and concentrated under reduced pressure to ˜100 mL. Hexanes (100 mL) were added and the precipitated was collected by filtration and washed with hexanes (2×50 mL) to give 14.76 g (84%) of the product as a colourless solid.

¹H-NMR (CDCl₃) δ: 1.62 (s, 3H); 2.16 (s, 3H); 6.40 (s, 2H); 7.10-7.40 (m, 15H).

### EXAMPLE 11

### (5R-3-(3-Fluoro-4-{6-[(2-propyl-1H-imidazol-1-yl)acetyl]pyridin-3-yl}phenyl)-5-(1H-1,2,3-triazol-1-ylmethyl)-1,3-oxazolidin-2-one

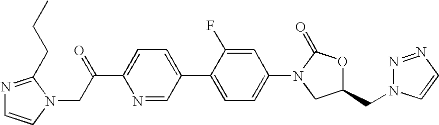

1-(5-Bromopyridin-2-yl)-2-(2-propyl-1H-imidazol-1-yl)ethanone (Intermediate 28, 255 mg, 0.825 mmol), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7, 384 mg, 0.99 mmol), potassium carbonate (343 mg, 2.48 mmol) and tetrakis (triphenylphospine) palladium (0) (46 mg, 0.04 mmol) were reacted like described under Example 1, but the reaction was heated at 80° C. for 30 minutes. Chromatography on silica gel with hexanes/acetone (1:1 to 0:1) gave 171 mg of the product an off-white solid, mp 81° C.

MS (ESP): 490 (MH⁺) for C₂₅H₂₄FN₇O₃

¹H-NMR (DMSO-d₆) δ: 0.89 (m, 3H); 1.63-1.70 (m, 2H); 2.88-2.93 (m, 2H); 3.44 (m, 2H), 3.98 (m, 1H), 4.25 (s, 1H); 4.96 (m, 1H); 5.24 (s, 1H); 6.09 (s, 2H); 7.59 (m, 1H); 7.70 (m, 2H); 7.77 (m, 2H); 8.12 (s, 1H); 8.19 (m, 1H); 8.26-8.28 (m, 1H), 9.03 (s, 1H).

### Intermediate 28: 1-(5-Bromopyridin-2-yl)-2-(2-propyl-1H-imidazol-1-yl)ethanone

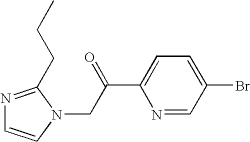

2-Bromo-1-(5-bromopyridin-2-yl)ethanone hydrobromide (Intermediate 9, 400 mg, 1.81 mmol) was suspended in dry THF (5 mL) and cooled to 0° C. 2-Propylimidazole (487 mg, 4.4 mmol) was added and the mixture was stirred for 2 hour. The reaction mixture was diluted with dichloromethane, washed with water, and dried over sodium sulfate. Chromatography on silica gel with dichloromethane/methanol (20:1) gave 256 mg of the product as an off-white solid.

MS (ESP): 309 (MH⁺) for C₁₃H₁₄BrN₃O

### EXAMPLE 12

### (5R-3-(3-Fluoro-4-{6-[(2-isopropyl-1H-imidazol-1-yl)acetyl]pyridin-3-yl}phenyl)-5-(1H-1,2,3-triazol-1-ylmethyl)-1,3-oxazolidin-2-one

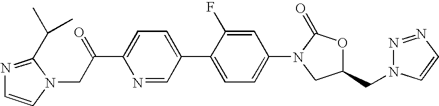

1-(5-Bromopyridin-2-yl)-2-(2-isopropyl-1H-imidazol-1-yl)ethanone (Intermediate 29, 255 mg, 0.825 mmoles), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7,384 mg, 0.99 mmol), potassium carbonate (343 mg, 2.48 mmol) and tetrakis (triphenylphospine) palladium (0) (46 mg, 0.04 mmol) were reacted and product was purified like described under Example 11, to give 174 mg of the product as an off-white solid, mp 82-83° C.

MS (ESP): 490 (MH⁺) for C₂₅H₂₄FN₇O₃

¹H-NMR (DMSO-d₆) δ: 1.21 (m, 6H); 3.39 (m, 1H); 3.95-4.00 (m, 1H), 4.28 (m, 1H), 4.86 (s, 2H); 5.17-5.20 (m, 1H); 6.13 (s, 2H); 7.45-7.48 (dd, 1H); 7.59 (m, 1H); 7.70 (m, 2H); 7.77 (m, 2H); 8.12 (s, 1H); 8.19 (m, 1H); 8.26-8.28 (m, 1H), 9.03 (s, 1H).

### Intermediate 29: 1-(5-Bromopyridin-2-yl)-2-(2-isopropyl-1H-imidazol-1-yl)ethanone

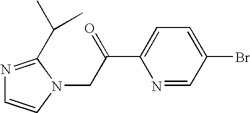

2-Bromo-1-(5-bromopyridin-2-yl)ethanone hydrobromide (Intermediate 9, 400 mg, 1.81 mmol) and 2-isopropyl imidazole (487 mg, 4.4 mmol) were reacted like described under Intermediate 28 to give 255 mg of the product as an off-white solid.

MS (ESP): 309 (MH⁺) for C₁₃H₁₄BrN₃O

### EXAMPLE 13

### (5R)-3-(4-{6-[(2,4-Dimethyl-1H-imidazol-1-yl)acetyl]pyridin-3-yl}-3-fluorophenyl)-5-(1H-1,2,3-triazol-1-ylmethyl)-1,3-oxazolidin-2-one

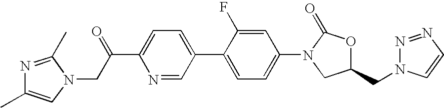

1-(5-Bromopyridin-2-yl)-2-2,4-dimethyl-1H-imidazol-1-yl)ethanone (Intermediate 30, 210 mg, 0.714 mmol), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7, 333 mg, 0.86 mmol), potassium carbonate (296 mg, 2.14 mmol) and tetrakis (triphenylphospine) palladium (0) (46 mg, 0.04 mmol) were reacted and product was purified like described under Example 11, to give 105 mg of the product as an off-white solid, mp 78° C.

MS (ESP): 476 (MH⁺) for C₂₄H₂₂FN₇O₃

¹H-NMR (DMSO-d6) δ: 2.26 (s, 3H); 3.55 (s, 3H); 3.95 (m, 1H), 4.27 (m, 1H); 4.98 (m, 2H); 5.18-5.22 (m, 1H); 5.98 (s, 2H); 7.27 (m, 1H); 7.43-7.64 (m, 2H); 7.76 (m, 2H); 8.10 (m, 1H); 8.19 (m, 1H); 8.26-8.28 (m, 1H), 9.03 (s, 1H).

### Intermediate 30: 1-(5-Bromopyridin-2-yl)-2-(2,4-dimethyl-1H-imidazol-1-yl)ethanone

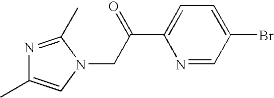

2-Bromo-1-(5-bromopyridin-2-yl)ethanone hydrobromide (Intermediate 9, 400 mg, 1.81 mmol) and 2,4 dimethyl imidazole (422 mg, 4.4 mmol) were reacted like described under Intermediate 28 to give 210 mg of the product as an off-white solid. MS (ESP): 280 (MH⁺) for C₁₁H₉BrN₃O

### EXAMPLE 14

### (5R)-3-(4-{6-[(2-Ethyl-1H-imidazol-1-yl)acetyl]pyridin-3-yl}-3-fluorophenyl)-5-(1H-1,2,3-triazol-1-ylmethyl)-1,3-oxazolidin-2-one

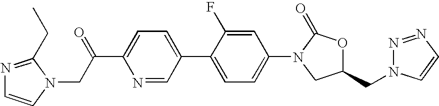

1-(5-Bromopyridin-2-yl)-2-(2-ethyl-1H-imidazol-1-yl)ethanone (Intermediate 31, 237 mg, 0.806 mmoles), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7, 376 mg, 0.97 mmol), potassium carbonate (340 mg, 2.41 mmol) and tetrakis (triphenylphospine) palladium (0) (46 mg, 0.04 mmol) were reacted and product was purified like described under Example 11, to give 174 mg of the product as an off-white solid, mp 77-79° C.

MS (ESP): 476 (MH⁺) for C₂₄H₂₂FN₇O₃

¹H-NMR (DMSO-d6) δ: 1.21 (m, 3H); 2.89-2.96 (m, 2H); 3.52 (m, 2H); 3.98-3.98 (m, 1H), 4.28 (m, 1H), 4.86 (s, 2H); 5.17-5.20 (m, 1H); 6.08 (s, 2H); 7.42-7.44 (dd, 1H); 7.57-7.72 (m, 1H); 7.76 (s, 1H); 7.98-8.08 (m, 1H); 8.19 (s, 1H); 8.43-8.59 (m, 1H); 8.82 (m, 2H).

### Intermediate 31: 1-(5-Bromopyridin-2-yl)-2-(2-ethyl-1H-imidazol-1-yl)ethanone

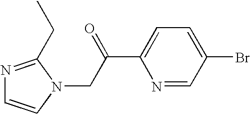

2-Bromo-1-(5-bromopyridin-2-yl)ethanone hydrobromide (Intermediate 9, 400 mg, 1.81 mmol) and 2-ethyl imidazole (427 mg, 4.4 mmol) were reacted like described under Intermediate 28 to give 237 mg of the product as an off-white solid.

MS (electrospray): 280 (MH⁺) for C₁₁H₉BrN₃O

### EXAMPLE 15

### (5R)-3-[3-Fluoro-4-(6-{[2-(methoxymethyl)-1H-imidazol-1-yl]acetyl}pyridin-3-yl)phenyl]-5-(1H-1,2,3-triazol-1-ylmethyl)-1,3-oxazolidin-2-one

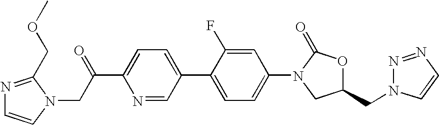

1-(5-Bromopyridin-2-yl)-2-[2-(methoxymethyl)-1H-imidazol-1-yl]ethanone (Intermediate 32, 400 mg, 1.3 mmol), (5R)-3-[3-fluoro-4-(4,4,5,5-tetramethyl-1,3,2-dioxaborolan-2-yl)phenyl]-[(1H-1,2,3-triazol-1-yl)methyl]oxazolidin-2-one (Intermediate 7, 582 mg, 1.5 mmol), potassium carbonate (539 mg, 3.9 mmol) and tetrakis (triphenylphospine) palladium (0) (75 mg, 0.065 mmol) were reacted and product was purified like described under Example 11, to give 150 mg of the product as an off-white solid, mp 78-80° C. MS (electrospray): 492 (M+1) for C₂₄H₂₂FN₇O₄

¹H-NMR (DMSO-d6) δ: 3.26 (s, 3H); 3.98 (m, 2H); 4.32 (s, 1H); 4.78 (s, 2H); 4.88 (m, 2H); 5.20 (s, 1H); 6.10 (s, 2H); 7.48 (m, 1H); 7.70 (m, 2H); 7.77 (m, 2H); 8.12 (s, 1H); 8.19 (m, 1H); 8.26-8.28 (m, 1H); 9.03 (s, 1H).

### Intermediate 32: 1-(5-Bromopyridin-2-yl)-2-[2-(methoxymethyl)-1H-imidazol-1-yl]ethanone

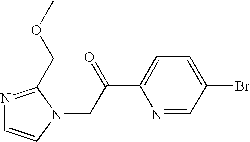

2-Bromo-1-(5-bromopyridin-2-yl)ethanone hydrobromide (Intermediate 9, 795 mg, 2.21 mmol) and 2-(methoxymethy)limidazole (192 mg, 1.7 mmol) were reacted like described under Intermediate 28 to give 340 mg of the product as an off-white solid.

MS (electrospray): 311 (MH⁺) for C₁₂H₁₂BrN₃O₂

What is claimed is:

1. A compound of the formula (I), or a pharmaceutically-acceptable salt, or pro-drug thereof, 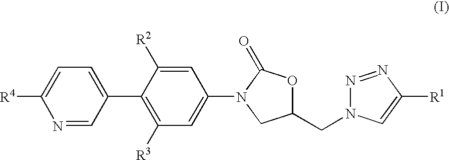
wherein:
R¹ is selected from hydrogen, halogen, cyano, methyl, cyanomethyl, fluoromethyl, difluoromethyl, trifluoromethyl, methylthio, and (2-4C)alkynyl;
R² and R³ are independently selected from hydrogen, fluoro, chloro and trifluoromethyl;
R⁴ is —C(O)R⁵; or
R⁴ is selected from —C(H)═N—OR⁸, —C(R⁵)═N—OH and —C(R⁵)═N—OR⁸;
R⁵is (1-6C)alkyl (substituted with 1 or 2 substituents independently selected from hydroxy, carboxy, (1-4C)alkoxy, HET-1 and NR⁶R⁷);
or R⁵ is (3-6C)cycloalkyl (optionally substituted with 1 substituent selected from hydroxy, carboxy, (1-4C)alkoxy and NR⁶R⁷);
or R⁵is HET-1;
R⁶ and R⁷ are independently selected from hydrogen, methyl, cyclopropyl (optionally substituted with methyl), carboxymethyl and (2-4C)alkyl (optionally substituted by a substituent selected from amino, (1-4C)alkylamino, di-(1-4C)alkylamino, carboxy, (1-4C)alkoxy and hydroxy);
or R⁶ and R⁷ together with a nitrogen to which they are attached form a 4, 5 or 6 membered, saturated or partially unsaturated heterocyclyl ring, optionally containing 1 further heteroatom (in addition to the linking N atom) independently selected from O, N and S, wherein a —CH₂-group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen to which R⁶ and R⁷are attached is not thereby quaternised) by 1 or 2 (1-4C)alkyl groups;
or R⁶ and R⁷ together with a nitrogen to which they are attached form an imidazole ring, which ring is optionally substituted on an available carbon atom by 1 or 2 (1-4C)alkyl (wherein a (1-4C)alkyl group is optionally substituted by methoxy or ethoxy);
R³ is (1-6C)alkyl (optionally substituted with 1 or 2 substituents independently selected from hydroxy, carboxy, (1-4C)alkoxy and NR⁶R⁷);
HET-1 is a 4, 5 or 6 membered saturated or partially unsaturated heterocyclyl ring, containing 1 or 2 heteroatoms independently selected from O, N and S, wherein a —CH₂-group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen is not thereby quaternised) by 1 or 2 (1-4C)alkyl.

2. A compound of formula (I) or a pharmaceutically-acceptable salt, or pro-drug thereof, as claimed in claim 1, wherein R¹ is selected from hydrogen, chloro, bromo, methyl and fluoromethyl.

3. A compound of formula (I) or a pharmaceutically-acceptable salt, or pro-drug thereof, as claimed in claim 1, wherein R² and R³ are independently selected from hydrogen and fluoro.

4. A compound of formula (I) or a pharmaceutically-acceptable salt, or pro-drug thereof, as claimed in claim 1, wherein R⁴ is —C(O)R⁵.

5. A compound of formula (I) or a pharmaceutically-acceptable salt, or pro-drug thereof, as claimed in claim 1, wherein R⁵ is (1-6C)alkyl (substituted with 1 or 2 substituents independently selected from hydroxy, carboxy, (1-4C)alkoxy, HET-1 and NR⁶R⁷).

6. A compound of formula (I) or a pharmaceutically-acceptable salt, or pro-drug thereof, as claimed in claim 1, which is a compound of formula (Ia):

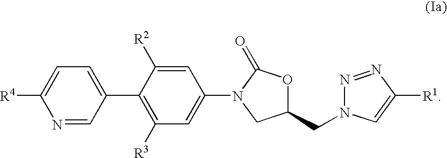

7. A pro-drug of a compound as claimed in claim 1.

8. A method for producing an antibacterial effect in a warm blooded animal said method comprising administering to said animal an effective amount of a compound of the invention as claimed in claim 1, or a pharmaceutically-acceptable salt, or in-vivo hydrolysable ester thereof.

9. A compound as claimed in claim 1, or a pharmaceutically-acceptable salt, or in-vivo hydrolysable ester thereof, for use as a medicament.

10. (canceled)

11. A pharmaceutical composition which comprises a compound as claimed in claim 1, or a pharmaceutically-acceptable salt or an in-vivo hydrolysable ester thereof, and a pharmaceutically-acceptable diluent or carrier.

12. A pharmaceutical composition as claimed in claim 11, wherein said composition comprises a combination of a compound of the formula (I) and an antibacterial agent active against gram-positive bacteria.

13. A pharmaceutical composition as claimed in claim 12, wherein said composition comprises a combination of a compound of the formula (I) and an antibacterial agent active against gram-negative bacteria.

14. A process for the preparation of a compound of formula (I) as claimed in claim 1 or a pharmaceutically acceptable salt or in-vivo hydrolysable ester thereof, which process comprises one of processes (a) to (m); and thereafter if necessary:
i) removing any protecting groups;
ii) forming a pro-drug (for example an in-vivo hydrolysable ester); and/or
iii) forming a pharmaceutically-acceptable salt;
wherein said processes (a) to (m) are as follows (wherein the variables are as defined in claim 1 unless otherwise stated):
a) modifying a substituent in, or introducing a substituent into another compound of the invention;
b) by reaction of one part of a compound of formula (II) (wherein X is a leaving group useful in palladium [0]coupling) with one part of a compound IIa, again with a leaving group X, such that the pyridyl-phenyl bond replaces the phenyl-X and pyridyl-X bonds; 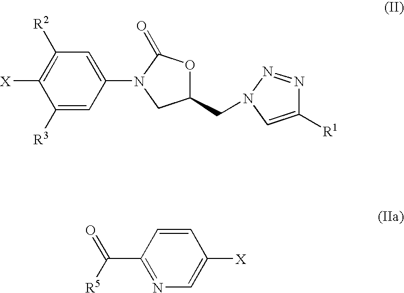
the leaving group X may be the same or different in the two molecules (II) and (IIa); 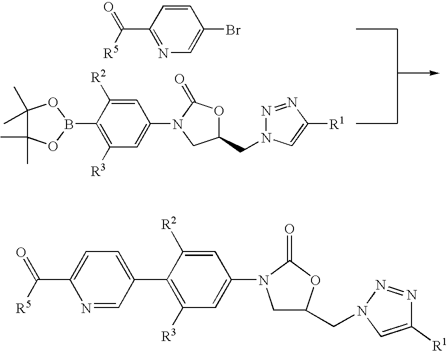
c) by reaction of a pyridyl-phenyl carbamate derivative (III) with an appropriately substituted oxirane to form an oxazolidinone ring; 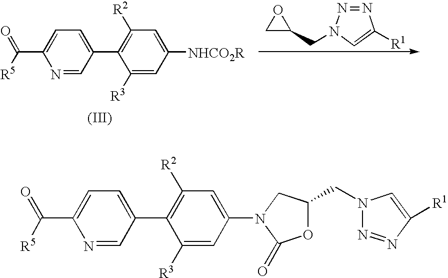
or by variations on this process in which the carbamate is replaced by an isocyanate or by an amine or/and in which the oxirane is replaced by an equivalent reagent X—CH₂CH(O— optionally protected)CH₂triazole-R¹ where X is a displaceable group; 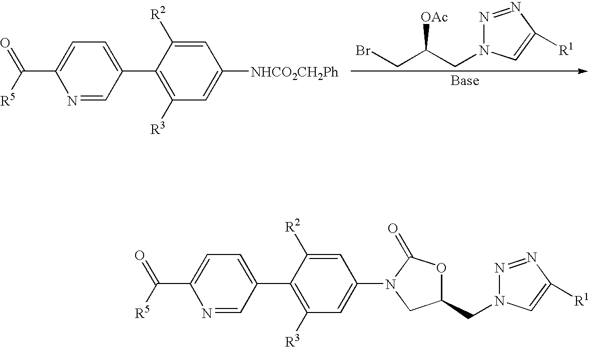
(d) for R⁴ as —COR⁵, by reaction of a compound of formula (IV): 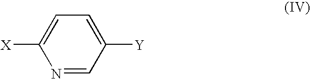
where X is a replaceable substituent and 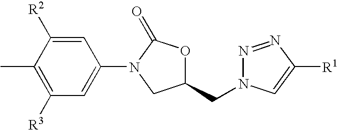
Y is halo or R with acylating agents; 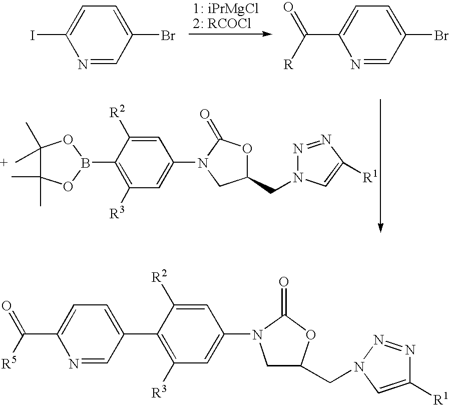
e) for R⁴ as —COR⁵, from an alpha halo ketone derivative by reaction with a nucleophile, to give a compound of formula (IIa), followed by reaction with a compound of formula (II), 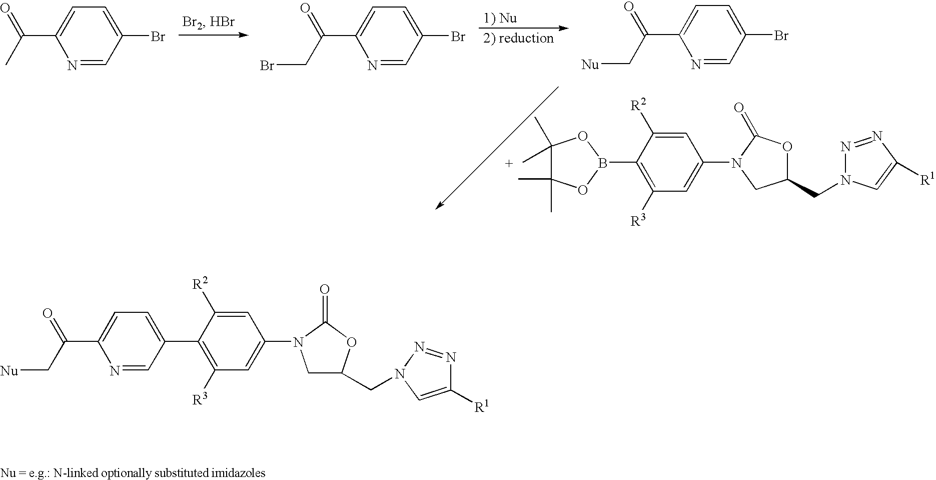
f) for R⁴ as —COR⁵, by oxidation of an alcohol derivative; 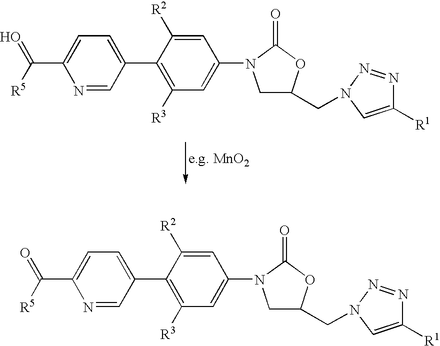
g) for R⁴ as an oxime, by reaction of an aldehyde or ketone with hydroxyl amine or a O-alkylated hydroxyl amine derivative: 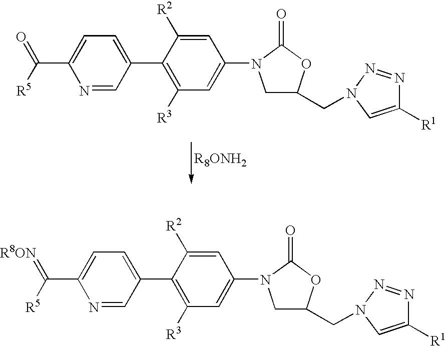
h) for R⁴ as —COR⁵, by reaction of a pyridyl-2-cyano derivative (V) with Grignard Reagents or similar metal alkyl reagents, followed by hydrolysis; 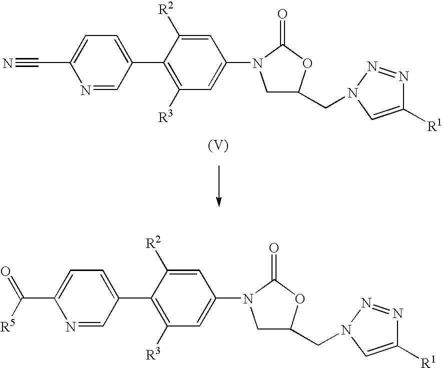
i) for R⁴ as —COR⁵, by alkylation of a pyridyl-2-carboxylate derivative of formula (IV) wherein X is a carboxylate derivative and Y is halo, followed by reaction with a compound of formula (II): 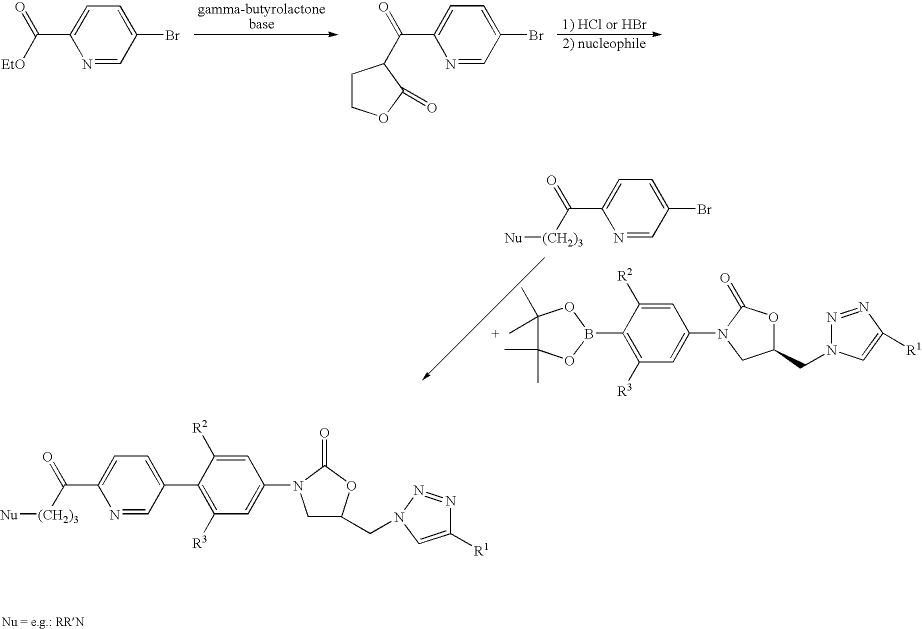
j) by formation of the triazole ring from a suitably functionalised intermediate in which the R⁴-pyridyl-phenyl ring system is already formed: 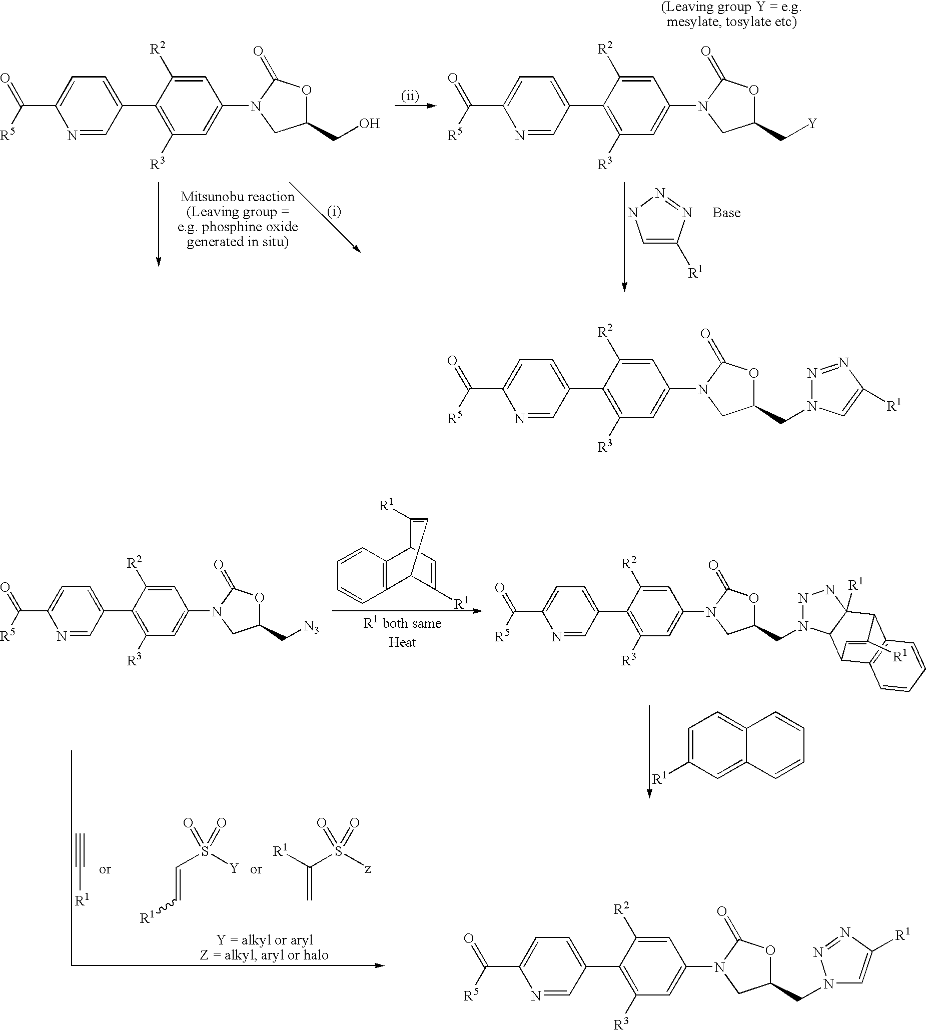
k) by cycloaddition via the azide to acetylenes; 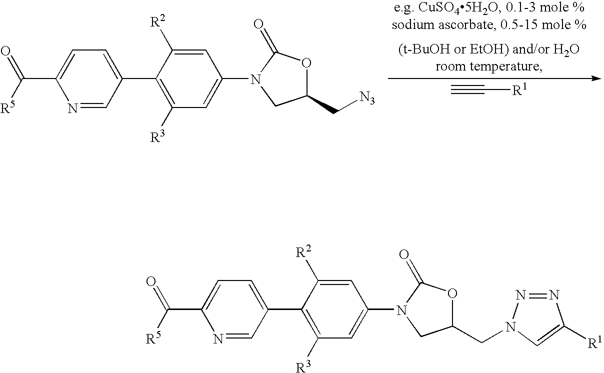
l) by reacting aminomethyloxazolidinones with 1,1-dihaloketone sulfonylhydrazones; 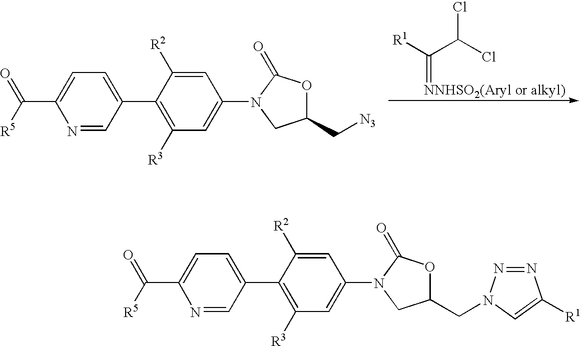
m) for R₁ as 4-halo, by reaching azidomethyl oxazolidinones with halovinylsulfonyl chlorides. [chemical structure]
